(19) United States
(12) Patent Application Publication
(10) Pub. No.: US 2017/0260242 A1
(43) Pub. Date: Sep. 14, 2017

(54) METHOD FOR MODIFYING RNA BINDING PROTIEN USING PPR MOTIF

(71) Applicants: Takahiro Nakamura, Fukuoka-shi (JP); Keiko Kobayashi, Fukuoka-shi (JP)

(72) Inventors: Takahiro Nakamura, Fukuoka-shi (JP); Keiko Kobayashi, Fukuoka-shi (JP)

(73) Assignee: Kyushu University, National University Corporation, Fukuoka-shi (JP)

(21) Appl. No.: 15/326,176

(22) PCT Filed: Mar. 11, 2011
(86) PCT No.: PCT/JP2011/055803

§ 371 Date: Jan. 13, 2017

(30) Foreign Application Priority Data
Mar. 11, 2010 (JP) 2010-055155

(51) Int. Cl. C07K 14/415

(52) U.S. Cl. CPC C07K 14/415

(57) ABSTRACT

The objects of the present invention are to identify the amino acids that play a principal role for the PPR motif to act as a RNA binding unit, as well as to provide a technology that regulates the RNA binding property thereof. The present invention provides a method for altering the RNA binding property of a PPR protein having one or more, preferably 2 or more, and more preferably 2-14 PPR motifs that consist of a polypeptide with a length of 30-38 amino acids, comprising a step of substituting one or more of the 1st, 4th, 8th, 9th, and 12th amino acids in the one or more PPR motifs with a different amino acid.

### TECHNICAL FIELD

The present invention relates to designing of a protein factor having various RNA binding properties that utilizes a polypeptide having a pentatricopeptide repeat (PPR) motif. The group of factors provided by the present invention can be employed for RNA regulation, and is useful in fields such as medical care and agriculture.

### BACKGROUND ART

In recent years, the function of RNA in organisms has come to be actively researched, and several RNA alteration technologies have been developed. For example, gene expression regulation (RNA interference) mediated by a small molecule RNA of 21-28 bases has begun to be actively utilized not only in the academic field, but also the medical care and agricultural fields as well as the industrial world.

In the meantime, RNA regulatory technology employing a protein factor has large expectations due to its broad application range with respect to the site and duration of action, etc. A pumilio protein is composed of a repeat of multiple puf motifs consisting of 38 amino acids. It has been shown that one puf motif binds to one RNA base (Non-Patent Literature 1), and a protein having novel RNA binding property employing a pumilio protein (Non-Patent Literature 2), as well as a technology for altering RNA binding property (Non-Patent Literature 3) have been attempted.

On the other hand, a novel protein that forms a large family of as many as 500 merely in plants, a pentatricopeptide repeat (PPR protein), has been identified from genome sequence information (Non-Patent Literatures 4 and 5). As the name indicates, a PPR protein is composed of repeats of 35 amino acids, and one unit thereof that is 35 amino acids is designated a PPR motif. The 500 PPR proteins each act on a different organellar RNA molecule to take part in almost every RNA metabolism such as cleaving, splicing, editing, stability, and translation. Most PPR proteins are composed only of a repeat of approximately ten PPR motifs, and in many cases the domain necessary for catalyzation cannot be found. For this reason, this molecule entity is thought to be a RNA adaptor (Non-Patent Literature 6).

### CITATION LIST

Non-Patent Literature 1: Wang, X., McLachlan, J., Zamore, P. D., and Hall, T. M. (2002). Modular recognition of RNA by a human pumilio-homology domain. Cell 110, 501-512.

Non-Patent Literature 2: Ozawa, T., Natori, Y., Sato, M., and Umezawa, Y. (2007). Imaging dynamics of endogenous mitochondrial RNA in single living cells. Nature Methods 4, 413-419.

Non-Patent Literature 3: Cheong, C. G., and Hall, T. M. (2006). Engineering RNA sequence specificity of Pumilio repeats. Proc. Natl. Acad. Sci. USA 103, 13635-13639.

Non-Patent Literature 4: Small, I. D., and Peeters, N. (2000). The PPR motif—a TPR-related motif prevalent in plant organellar proteins. Trends Biochem. Sci. 25, 46-47.

Non-Patent Literature 5: Lurin, C., Andres, C., Aubourg, S., Bellaoui, M., Bitton, F., Bruyere, C., Caboche, M., Debast, C., Gualberto, J., Hoffmann, B., Lecharny, A., Le Ret, M., Martin-Magniette, M. L., Mireau, H., Peeters, N., Renou, J. P., Szurek, B., Taconnat, L., and Small, I. (2004). Genome-wide analysis of Arabidopsis pentatricopeptide repeat proteins reveals their essential role in organelle biogenesis. Plant Cell 16, 2089-2103.

Non-Patent Literature 6: Chory, J., and Woodson, J. D. (2008). Coordination of gene expression between organellar and nuclear genomes. Nature Rev. Genet. 9, 383-395.

### SUMMARY OF THE INVENTION

### Problems to be Solved by the Invention

Biological species to which RNA interference can be applied are limited to several eukaryotes due to the necessity of many protein factors that eukaryotes innately possess. Moreover, there are several restrictions as a gene expression regulatory technology including e.g. it can only work in the direction of inhibiting gene expression and the duration of action is short because it is a RNA component.

Moreover, in the RNA regulatory technology employing a protein factor, the correlation between the amino acid sequence configuring the protein and RNA affinity, as well as rules of RNA sequences that can bind to the amino acid sequences are virtually unclear. The pumilio protein exists as an exception, but motifs that belong to the puf family are highly conserved in amino acid sequences between each other, and there are only a small number in existence. For this reason, there is a problem that it can only be employed for constructing protein factors that act on limited RNA sequences.

The nature of the PPR protein as a RNA adaptor is anticipated to be determined by the nature of each PPR motif that configure the PPR protein and the nature that is exerted by a combination thereof. However, the PPR motif is identified with a computational science method of genome sequence information, and the correlation between its amino acid sequence and function was completely unclear. If amino acids essential for the PPR motif to exert RNA binding property were identified and a method for regulating binding property was established, there is a possibility that a novel protein that can bind to RNA molecules having various sequences and lengths can be designed by alteration of the PPR motif or alteration of a combination thereof.

Accordingly, the problems set by the present inventors were to identify the amino acids that play a principal role for the PPR motif to act as a RNA binding unit, as well as to provide a technology that regulates the RNA binding property thereof. If a protein factor having various RNA binding properties that utilizes a PPR motif can be provided, it may become a universal technology that can be utilized in various scenes.

Means for Solving the Problems

In order to solve the above problems, the present inventors prepared multiple recombinant mini PPR proteins composed of two PPR motifs and identified a PPR motif having different RNA binding property. Further, amino acids necessary for the PPR motif to exert RNA binding ability were identified by comparing the RNA binding property and amino acid sequence thereof as well as performing amino acid substitution. Then, by substituting such amino acids, the present inventors succeeded in altering the RNA binding property thereof (to improve or reduce RNA binding activity.)

According to investigations by the present inventors, among the two α helix structures that configure the motif, the 1st, 4th, 8th, and 12th amino acids that configure the first helix (Helix A) are particularly involved in the RNA binding property of the PPR motif, and it was found that by focusing on these amino acids, a PPR motif having a different RNA binding property or a novel protein having such a motif can be configured.

The present invention provides the following.
[1] A method for altering the RNA binding property of a PPR protein having one or more (preferably 2 or more, more preferably 2-14) PPR motifs that consist of a polypeptide with a length of 30-38 amino acids represented by Formula I:

[Chemical Formula 1]

—Xᵢ-(Helix A)-Xii-(Helix B)-Xᵢᵢᵢ- (Formula I)

(wherein:

Helix A is a portion with a length of 12 amino acids that can form an α helix structure, Helix A is represented by Formula II:

[Chemical Formula 2]

-A₁-A₂-A₃-A₄-A₅-A₆-A₇-A₈-A₉-A₁₀-A₁₁-A₁₂- (Formula II)

(A₁-A₁₂ each independently represent an amino acid);

Helix B is a portion with a length of 11-13 amino acids that can form an α helix structure; and

Xᵢ₋ᵢᵢᵢ are each independently a portion consisting of a length of 1-9 amino acids or does not exist,)

comprising a step of substituting one or more amino acids selected from the group consisting of A₁, A₄, A₈, A₉, and A₁₂ (preferably the group consisting of A₁, A₄, A₈, and A₁₂) in the one or more PPR motifs with a different amino acid.
[2] A method according to 1 wherein the method is for improving the RNA binding activity of the PPR protein, the PPR protein has two or more PPR motifs, and the method comprises any of the following steps of:

a substitution to make A₁ of the first PPR motif a basic amino acid, preferably arginine;

a substitution to make A₄ of the second PPR motif a neutral amino acid, preferably threonine;

a substitution to make A₈ of the first PPR motif a basic amino acid, preferably lysine, or an acidic amino acid, preferably aspartic acid; and

a substitution of A₁₂ of the first PPR motif and/or A₁₂ of the second PPR motif to make either one a basic amino acid and the other a neutral amino acid or a hydrophobic amino acid.
[3] A method according to [1] or [2] comprising an alteration that considers the following in the one or more PPR motifs:

cooperation between A₁ of a motif and A₄ of the same motif, and/or

cooperation between A₈ of a motif and A₁₂ of the same motif.
[3-1] A method according to [1] wherein the method is for improving the RNA binding activity of the PPR protein, and the method comprises any of the following steps of:

a substitution to make A₁ of the first PPR motif a basic amino acid, preferably arginine;

a substitution to make A₄ of the second PPR motif a neutral amino acid, preferably threonine;

a substitution to make A₈ of the first PPR motif a basic amino acid, preferably lysine, or an acidic amino acid, preferably aspartic acid; and

a substitution of A₁₂ of the first PPR motif and/or A₁₂ of the second PPR motif to make either one a basic amino acid and the other a neutral amino acid or a hydrophobic amino acid.
[3-2] A method according to [1] wherein the method is for improving the RNA binding activity of the PPR protein, and the method comprises the following step of:

a substitution of A₈ of the first PPR motif and/or A₈ of the second PPR motif to make both basic amino acids or acidic amino acids, or either one a basic amino acid and the other an acidic amino acid.
[3-3] A method according to [1] wherein the method is for reducing the RNA binding activity of the PPR protein, and the method comprises the following step of:

a substitution of A₈ of the first PPR motif and/or A₈ of the second PPR motif to make at least one a neutral amino acid or a hydrophobic amino acid.
[4] A method for designing a protein having RNA binding property that employs a PPR motif according to 1 that has a basic or acidic amino acid at A₈ and A₁₂.
[4-1] A method for designing a protein having RNA binding property that utilizes a sequence represented by Formula II:

[Chemical Formula 3]

-A₁-A₂-A₃-A₄-A₅-A₆-A₇-A₈-A₉-A₁₀-A₁₁-A₁₂- (Formula II)

(wherein A₁ is a basic amino acid, preferably arginine;

A₄ is a neutral amino acid, preferably threonine;

A₈ is a basic amino acid, preferably lysine, or an acidic amino acid, preferably aspartic acid; and

A₁₂ is a basic or neutral amino acid or hydrophobic.)
[5] A method for designing a protein that can specifically bind to a target base in a RNA, comprising employing a PPR motif according to 1, wherein A₁ and A₄ is the combination to which the target base is specifically bound to improve the base binding specificity of that motif.
[6] A method according to [5], wherein the target base is adenine, uracil, or guanine (preferably adenine or uracil, and more preferably adenine), and the combination of A₁ and A₄ is valine and threonine or isoleucine and threonine; or

the target base is adenine, guanine, or uracil (preferably adenine or guanine, and more preferably adenine), and the combination of A₁ and A₄ is valine and asparagine, isoleucine and asparagine, or alanine and asparagine in that order; or

the target base is guanine, thymine, or adenine (preferably guanine or thymine, and more preferably guanine), and the combination of A₁ and A₄ is leucine and asparagine in that order.
[7] A method according to [5] or [6], comprising employing PPR motifs corresponding to each of two or more target bases in a RNA, wherein

A₁ and A₄ in one motif is the combination to which the corresponding target base is specifically bound to improve the binding specificity of that motif; and

A₁ and A₄ in another motif is the combination to which the corresponding target base is specifically bound to improve the base binding specificity of each motif.
[7-1] A PPR motif according to [1] (wherein A₁ is a basic amino acid, preferably arginine;

A₄ is a neutral amino acid, preferably threonine;

A₈ is a basic amino acid, preferably lysine, or an acidic amino acid, preferably aspartic acid; and

A₁₂ is a basic or neutral amino acid or hydrophobic.)
[8] A protein comprising all or a portion with RNA binding activity of a polypeptide consisting of an amino acid sequence of SEQ ID NOs. 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 168, 170, 172, or 174.
[9] A polynucleotide encoding a RNA binding protein according to [8].
[10] A polynucleotide (DNA or RNA) according to 9 having a base sequence of SEQ ID NOs. 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 169, 171, 173, or 175.

A method for regulating RNA function that employs a PPR protein altered with a method according to any one of [1] to [3], a protein designed with a method according to any one of [4] to [7], or a protein according to [8].

### Effects of the Invention

By virtue of the present invention, the binding activity of a protein having RNA binding property with a RNA can be increased, or adversely, the binding activity can be reduced. When the protein is an enzyme, a rise in the dissociation rate (increase in reaction frequency) with the substrate RNA can be expected.

Moreover, by virtue of the present invention, a novel protein having RNA affinity and binding RNA base selectivity that differ from natural PPR proteins can be provided.

Further, the PPR motif or PPR protein provided by the present invention is useful for preparing a conjugated protein.

Further, by virtue of the present invention, a polynucleotide (a gene, a DNA, or a RNA) encoding such a protein is provided that can be utilized for creating transformants or for imparting regulations or functions for organisms (cells, tissues, or individuals) in various scenes.

Further, by virtue of the present invention, a method for designing a protein that has binding specificity to bases in a RNA or to a desired RNA is provided.

### BRIEF DESCRIPTION OF THE DRAWINGS

FIG. 1 shows the schematic diagram and RNA binding activity of mini PPR proteins.

FIG. 2 shows the RNA binding activity and amino acid sequence of mini PPR proteins.

FIG. 3-1 shows the RNA binding activity of mini PPR proteins with amino acid substitution.

FIG. 3-2 shows the RNA binding activity of mini PPR proteins with amino acid substitution.

FIG. 3-3 shows the RNA binding activity of mini PPR proteins with amino acid substitution.

FIG. 4 shows the schematic diagram of introducing amino acid substitution and the RNA binding activity of mini PPR proteins with amino acid substitution.

FIG. 5 shows the RNA binding activity of mini PPR proteins having the 12th amino acid substituted.

FIG. 6 shows the RNA binding activity of mini PPR proteins having the 8th amino acid substituted.

FIG. 7 shows the composition of amino acids configuring the PPR motif.

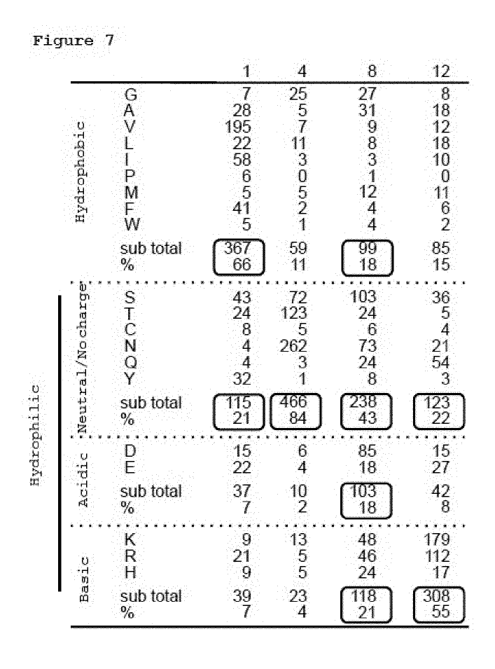

FIG. 8 shows the association between the 1st, 2nd, 4th, and 8th amino acids.

FIG. 9 shows the phase of acidic or basic amino acids at the 1st, 4th, 8th, 9th, and 12th positions in each PPR motif in PPR proteins.

FIG. 10 shows the binding specificity of mini PPR proteins against RNA.

FIG. 11 shows the polymorphism between potential HCF152 homologous proteins.

FIG. 12-1 shows the comparison of amino acid sequences of potential HCF152 homologous proteins in various plants (At is Arabidopsis HCF152, and potential HCF152 homologous protein sequences for Vv1 (Vitis vinifera), Vv2 (Vitis vinifera), Rc (Ricinus communis), Pt (Populus trichocarpa), Sb (Sorghum bicolor), and Os (Oryza sativa)). The lines over the sequence show PPR motifs, and amino acids (1st, 4th, 8th, and 12th) involved in RNA interaction are shown in gray. Moreover, Helix shows the secondary structure of proteins (helix, h; coil region, c; and β sheet, e) and AAP shows the number of amino acid polymorphisms.

FIG. 12-2 is continued from FIG. 12-1.

FIG. 13 shows the binding specificity of proteins composed of one PPR motif against RNA.

FIG. 14-1 shows the amino acid or base sequences related to the present invention.

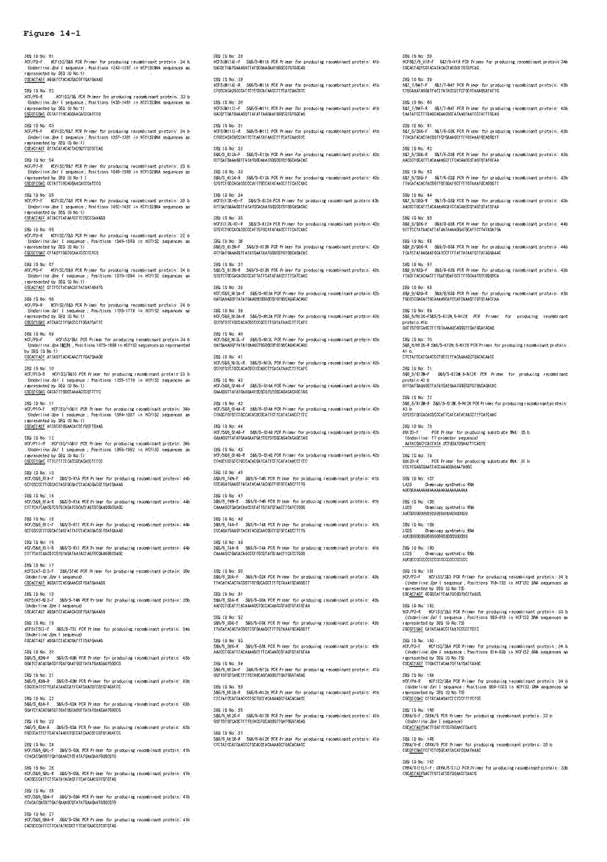

FIG. 14-2 shows the amino acid or base sequences related to the present invention.

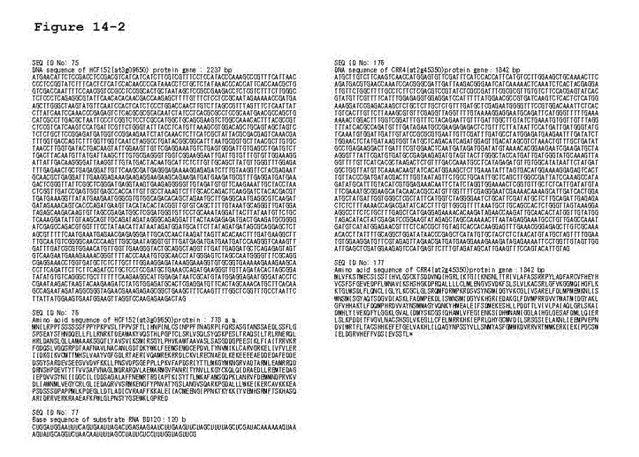

FIG. 14-3 shows the amino acid or base sequences related to the present invention.

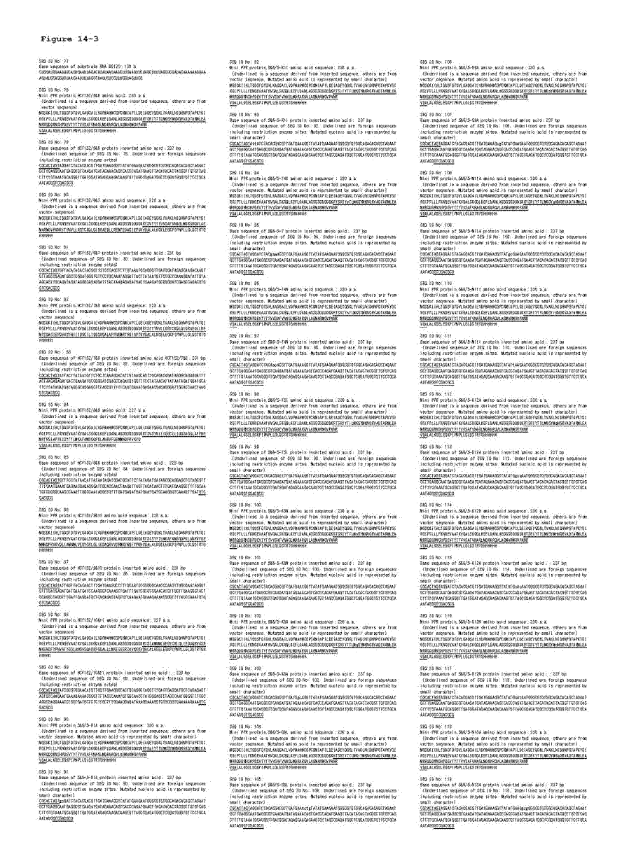

FIG. 14-4 shows the amino acid or base sequences related to the present invention.

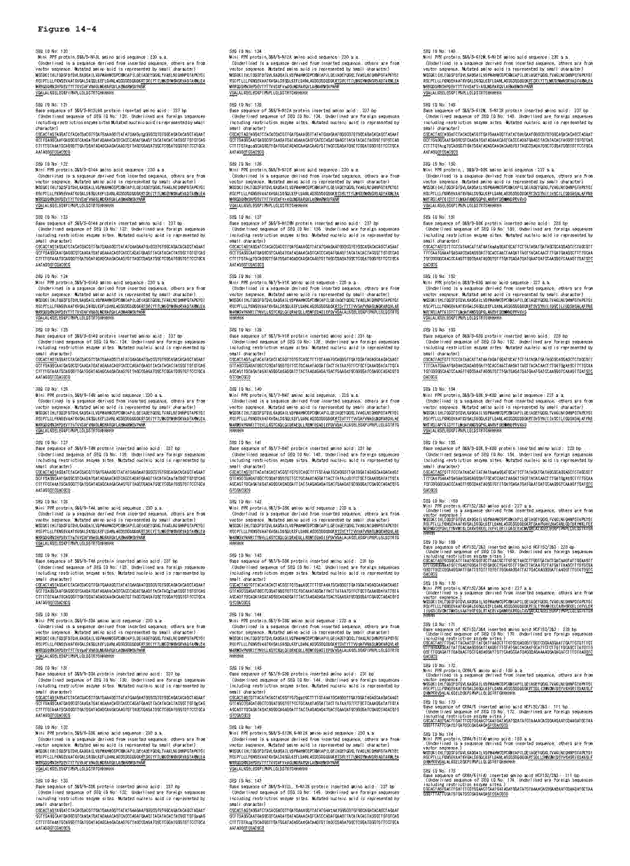

FIG. 14-5 shows the amino acid or base sequences related to the present invention.

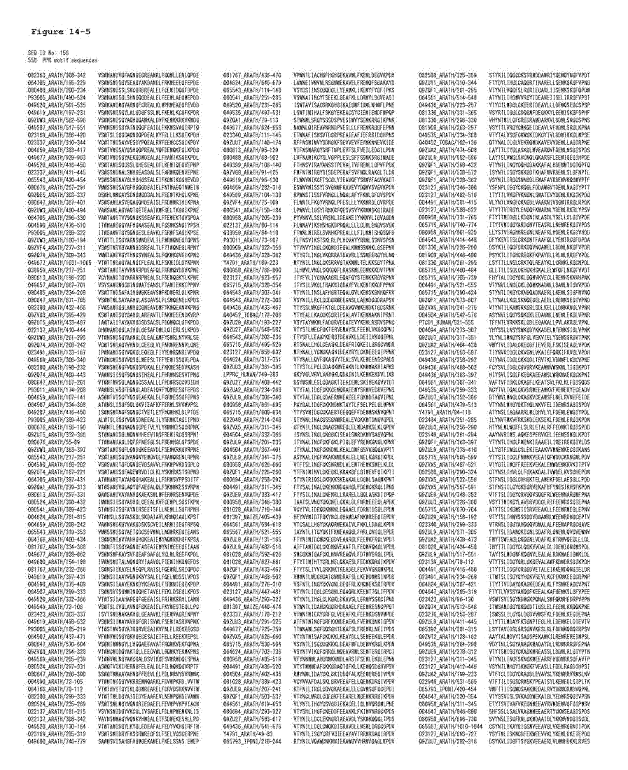

FIG. 14-6 shows the amino acid or base sequences related to the present invention.

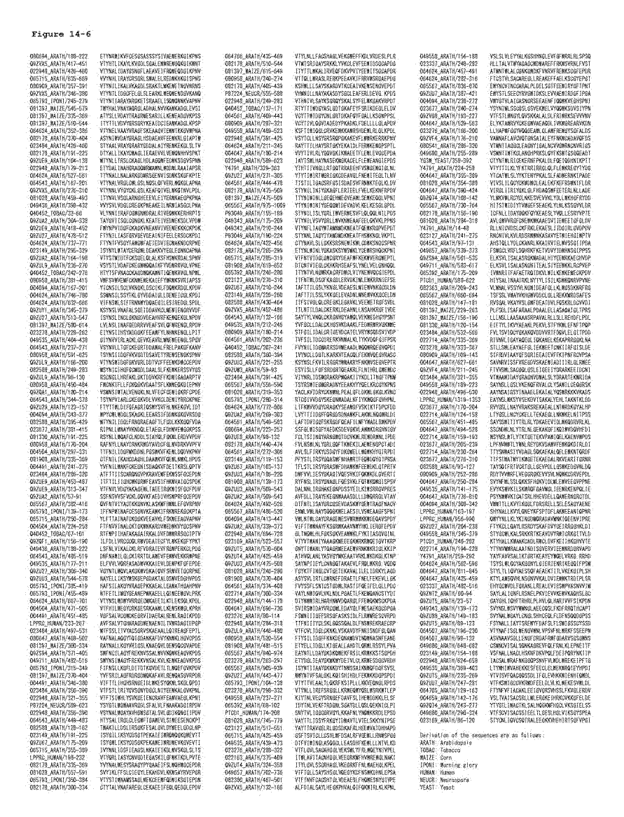

### DESCRIPTION OF EMBODIMENTS

A “PPR motif” as referred to herein, unless otherwise particularly described, is a polypeptide composed of 30-38 amino acids having an amino acid sequence of which the E value obtained when the amino acid sequence is analyzed in a protein domain search program on the web with PF01535 for Pfam, IPR002885 for InterProScan, and PS51375 for Prosite is a given value or less (desirably E-03). The position of the amino acids configuring the PPR motif defined by the present invention is synonymous with PF01535 and IPR002885, but is two less than the amino acid position for PS51375 (e.g. position #1 in the present invention is #3 in PS51375).

Web Information:

Pfam: http://pfam.sanger.ac.uk/InterProScan: http://www.ebi.ac.uk/Tools/InterProScan/Prosite: http://www.expasy.org/prosite/

The conserved amino acid sequence of a PPR motif is shown in the aforementioned Non-Patent Literatures 4 and 5. Conservation in the amino acid level is low, but the two α helices are well conserved in the secondary structure. The PPR motifs consist of 30-38 amino acids and have variable lengths, but a typical PPR motif is composed of 35 amino acids.

The PPR motif according to the present invention preferably consists of the following structure:

[Chemical Formula 4]

-A₁-A₂-A₃-A₄-A₅-A₆-A₇-A₈-A₉-A₁₀-A₁₁-A₁₂- (Formula II)

(wherein:

Helix A is a portion with a length of 12 amino acids that can form an α helix structure, Helix A is represented by Formula II:

[Chemical Formula 5]

-A₁-A₂-A₃-A₄-A₅-A₆-A₇-A₈-A₉-A₁₀-A₁₁-A₁₂- (Formula II);

Helix B is a portion with a length of 11-13 amino acids that can form an α helix structure; and

Xᵢ₋ᵢᵢᵢ are each independently a portion consisting of a length of 1-9 amino acids or does not exist.) Aₓ represents an amino acid. Note that the 1st amino acid (A₁) may or may not be contained in the α helix structure. The amino acids to be the skeleton for the α helix structure are designated A₃, A₆, A₇, and A₁₀.

A “PPR protein” as referred to herein, unless otherwise particularly described, refers to a PPR protein having 1 or more, preferably 2 or more, and more preferably 2-14 of the PPR motifs described above. In particular, a protein having two PPR motifs may be referred to herein as a “mini PPR protein.” A “protein” as referred to herein, unless otherwise particularly described, refers to all substances that consist of a polypeptide (a chain where multiple amino acid are bound by peptide binding), and also includes those consisting of a relatively small molecule polypeptide.

The “binding property” in reference to the binding ability of a protein with a RNA as referred to herein, unless otherwise particularly described, is used as a concept encompassing binding activity and binding specificity. Unless otherwise particularly described, “binding activity” is employed synonymously herein with “affinity,” and refers to the strength of binding. The presence or absence or the extent of the binding activity can be appropriately determined by those skilled in the art with various technologies used for similar objectives, and the Examples herein describe in detail the gel shift method for this purpose. “No” binding activity in reference to a protein as referred to herein refers to the case where the disassociation constant (Kd) cannot be calculated even with 3750 nM of protein. As referred to herein, a protein has “binding specificity” in reference to a RNA base, unless otherwise particularly described, refers to the fact that the binding activity against any one of the RNA bases is higher than the binding activity against others. A RNA base is a base among nucleic acid bases that configures a RNA, and specifically refers to adenine (A), guanine (G), cytosine (C), or uracil (U). Note that a protein designed by the present invention may have binding specificity against bases in a RNA, but does not necessarily bind to a nucleic acid monomer. As referred to herein, a protein has “binding specificity” in reference to a RNA, unless otherwise particularly described, refers to the fact that the binding activity against a RNA consisting of a base sequence is higher than the binding activity against a RNA having a different base sequence. Such a protein may have e.g. multiple PPR motifs which have binding specificity against each of the multiple bases in the target RNA. The presence or absence or the extent of the binding specificity of a protein against a RNA base or RNA can be appropriately determined by those skilled in the art, and the Examples herein describe in detail the gel shift method for this purpose. Having binding specificity against a subject is sometimes referred to as being able to recognize a subject or being able to identify a subject.

“Alteration” of binding property or binding activity is a concept encompassing improvement and reduction. Improving the binding activity refers to making Kd to 1/10 or less, and reducing refers to making Kd to 10 folds or more. Kd may differ depending on the RNA to be bound. For comparison purpose, those described in the Examples herein can be employed as standard RNA.

An “acidic amino acid” as referred to herein, unless otherwise particularly described, refers to an amino acid wherein the side chain (sometimes expressed as R group) has a negative charge at pH 7.0. Examples thereof are aspartic acid and glutamic acid.

A “basic amino acid” as referred to herein, unless otherwise particularly described, refers to an amino acid wherein the side chain has a positive charge at pH 7.0. Examples thereof are lysine, arginine, and histidine.

A “neutral amino acid” as referred to herein, unless otherwise particularly described, refers to an amino acid that is neither an acidic amino acid nor a basic amino acid. Examples thereof are asparagine, serine, glutamine, threonine (sometimes expressed as threonine), glycine, tyrosine, tryptophan, cysteine, methionine, proline, phenylalanine, alanine, valine, leucine, and isoleucine.

A “hydrophobic amino acid” as referred to herein, unless otherwise particularly described, refers to an amino acid having a nonpolar aliphatic side chain. A hydrophobic amino acid is ordinarily employed synonymously with a nonpolar amino acid. Examples of a hydrophobic amino acid are glycine, tryptophan, methionine, proline, phenylalanine, alanine, valine, leucine, and isoleucine.

An “amino acid” as referred to herein may refer to a free amino acid or may refer to an amino acid residue that configures a peptide chain. Which meaning the term is employed in is clear to those skilled in the art from the context.

When referring to “substitution” herein in reference to the amino acid sequence of a motif or a protein, the means therefor is not particularly limited. Means for preparing a polynucleotide related to an amino acid sequence comprising substitution includes e.g. site-directed mutagenesis method (Kramer W & Fritz H-J: Methods Enzymol 154:350, 1987). Moreover, those skilled in the art can refer to the description in the Examples herein.

The present invention relates to a substitution of an amino acid at a particular position in a motif or protein. Substitution is not limited to a position defined as the present invention, and substitution to an amino acid with like natures can also occur at other positions in a motif or protein, and those comprising such a substitution are also encompassed in the scope of the present invention. Substitution to an amino acid with like natures refers to e.g. a substitution among acidic amino acids, a substitution among basic amino acids, a substitution among neutral amino acids, and a substitution among hydrophobic amino acids. The number of amino acids substituted in this respect is not particularly limited as long as the polypeptide that consists of that amino acid sequence has the desired function, and is for example about 1-9 or 1-4.

Although the method for searching a conserved amino sequence as the PPR motif has been established, no methods related to the amino acids necessary for expressing RNA binding property have been found before the present invention. The following knowledge is provided by virtue of the present invention:
(1) From the amino acid sequence of the PPR motif and preliminary structural prediction, it is predicted that amino acids that contribute to RNA binding are allocated in Helix A.
(2) Introduction of substitution into the five amino acids A₁, A₄, A₈, A₉, and A₁₂ may result in alteration of RNA binding property.
(3) A₁, A₄, and A₈ of the first (upstream) PPR motif act actively on the binding with RNA. In other words, by appropriately manipulating A₁, A₄, and A₈, the RNA affinity of the PPR motif and in turn the PPR protein may be improved.
(4) A₁₂ also acts actively on the RNA affinity of the PPR motif, and when multiple PPR motifs are involved, improvement of RNA affinity can be expected by appropriately combining a motif where the 12th amino acid is a basic amino acid and a motif where the same is a neutral (or hydrophobic) amino acid.
(5) Moreover, in a PPR protein having numerous (e.g. 4 or more, preferably 4-14, and more preferably 7-14) PPR motifs, A₈ is a basic or acidic amino acid in every other or every two of the multiple PPR motifs and/or A₁₂ is a basic or acidic amino acid in every other or every two of the multiple PPR motifs, and improvement of RNA binding property can be expected by mimicking such an allocation.
(6) There is a possibility that A₁ in a PPR motif and A₄ in the same PPR motif cooperate in RNA binding, and there is also a possibility that A₈ in a PPR motif and A₁₂ in the same PPR motif cooperate in RNA binding.

By virtue of the present invention, the affinity of an existing PPR protein can be altered.

Many of the PPR proteins are present in plants. For example, a type of PPR protein acts on pollen (male gamete) formation, and the RNA affinity thereof can be altered to elevate pollen formation efficiency. Moreover, since existing PPR proteins often act in the mitochondria or the chloroplast, alteration of the RNA affinity of the PPR protein may result in alteration of the function of mitochondria or chloroplast (it is known that photosynthesis, respiration, and synthesis of useful metabolites are changed due to PPR protein defect.) In animals, since it is known that a PPR protein defect identified as LRPPRC causes Leigh syndrome French Canadian (LSFC; Leigh syndrome, subacute necrotizing encephalomyelopathy), the present invention may contribute to the treatment (prevention, therapy, and suppression of progression) of LSFC.

The altered PPR motif or PPR protein obtained by the present invention can be linked with other functional proteins to be utilized as a useful conjugated protein. For example, one PPR protein has a RNA cleaving domain linked after the PPR motif repeat. In this way, by linking a RNA binding domain, a sequence specific RNA cleaving enzyme (RNA version of a restriction enzyme) may be configured. Moreover, GFP (green fluorescent protein) may be linked to be employed for visualizing the RNA of interest. Further, ribosome S1 protein is linked to expect improvement of translation speed.

In the meantime, among existing PPR proteins, there are some that act on DNA. Some of such PPR proteins are localized in the nucleus, and have a domain that interacts with Po12 (RNA transcription enzyme that exists in the nucleus) added thereto. Accordingly, such a domain can be linked to the PPR motif or PPR protein obtained by the present invention to aim for activation of transcription.

Moreover, PPR proteins include those that are known to act on the assignment of editing site in RNA editing (conversion of genetic information on the RNA; in many cases C→U.) This type of PPR protein has a domain that is anticipated to interact with a RNA editing enzyme called an E domain added at the C-terminal. By linking such E domain, base polymorphism is introduced, or contribution to the treatment of a disease or condition related to base polymorphism may be made.

The present invention provides a novel PPR protein, i.e. a protein comprising all or a portion with RNA binding activity of a polypeptide consisting of an amino acid sequence of SEQ ID NOs. 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 168, 170, 172, or 174. Also provided is a polynucleotide (DNA or RNA) encoding such a RNA binding protein, i.e. a polynucleotide having a base sequence of SEQ ID NOs. 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 169, 171, 173, or 175. Such a protein and polynucleotide may be synthesized or may be a natural product, and those skilled in the art are able to prepare them with a preexisting method.

The present invention relates to a substitution of an amino acid at a particular position in a motif or protein. By such a substitution, a protein that specifically binds to any RNA base or a RNA having any sequence can be designed.

The following knowledge is further provided by virtue of the present invention.

(7) In the comparison of amino acid sequences of homologous PPR proteins, when the 1st amino acid of a PPR motif is isoleucine and the 4th amino acid is asparagine, polymorphism of valine and alanine was seen in the 1st amino acid; and when the 1st amino acid is valine and the 4th amino acid is threonine, polymorphism of isoleucine was seen in the first amino acid. Accordingly, it is suggested that these polymorphic amino acids are amino acids that are responsible for the same function.

(8) When the 1st and 4th amino acids of a PPR motif are valine and threonine or isoleucine and threonine, that motif may have the binding specificity of binding strongly to A, then to U, and then to G.

(9) When the 1st and 4th amino acids of a PPR motif are valine and asparagine, isoleucine and asparagine, or alanine and asparagine, that motif may have specificity that binds strongly to A, then to G, and then to U.

(10) When the 1st and 4th amino acids of a PPR motif are leucine and asparagine, that motif may have specificity that binds strongly to G, then to T, and then to A.

(11) In a protein composed of one PPR motif, those having isoleucine and asparagine as the 1st and 4th amino acids (such as CAR4/6) has a preference to bind to A, and those where the 1st amino acid is altered to leucine and having leucine and asparagine as the 1st and 4th amino acids (such as CAR4/6 (I1A)) do not bind to A but bind well to G. In other words, by employing a PPR motif corresponding to the RNA recognition code for each of the 1st and 4th amino acids, a protein that binds to each of the bases can be prepared, and moreover, construction of a protein that binds to a RNA sequence having consecutive aforementioned bases is possible by linking.

Accordingly, by virtue of the present invention, a method for designing a protein that can specifically bind to any target RNA base and a method for designing a protein that can specifically bind to a RNA having any target sequence are provided.

### EXAMPLES

### Example 1

### Preparation of Mini PPR Protein Consisting of Two PPR Motifs]

(Preparation of Genome DNA from Arabidopsis Thailand)

Arabidopsis Thailand (ECT Columbia) was cultured for three weeks in a Murashige & Skoog medium (comprising 2% sucrose and 0.5% Gellangam). Green leaves (0.5 g) of the cultured plant were extracted by phenol/chloroform extraction, and then ethanol was added to insolubilize DNA. The DNA collected was dissolved in 100 μl of TE solution (10 mM Tris-hydrochloric acid (pH 8.0), 1 mM EDTA), 10 units of RNase A (DNase-free, TAKARA BIO INC.) was added, and this was reacted at 37° C. for 30 minutes. Then, the reaction solution was extracted again with phenol/chloroform extraction, after which the DNA was collected by ethanol precipitation. Ten micrograms of DNA were obtained.

### (Cloning of Gene Encoding Mini PPR Protein HCF152/5&6)

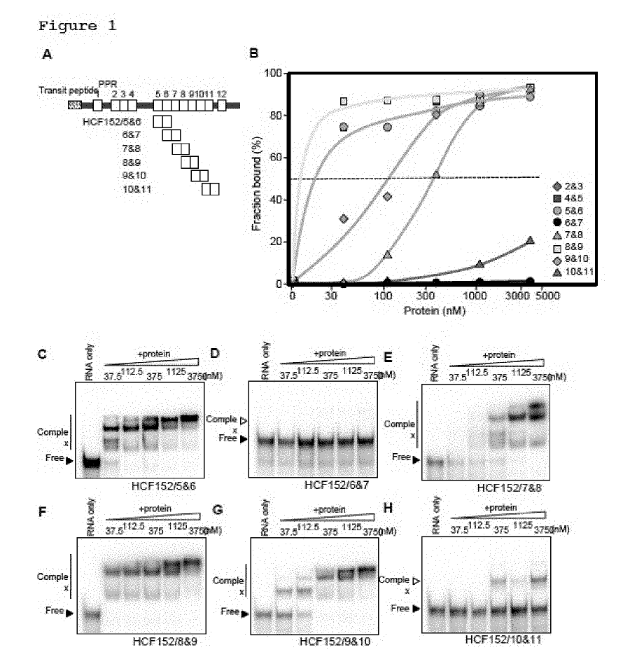

Preparation of genome DNA from Arabidopsis Thailand was carried out with the method described in Example 1 above. PPR protein gene HCF152 (at3g09650; SEQ ID NOs. 75 and 76) have twelve PPR motifs (FIG. 1A) (see Literature 1). Referring to the sequence information from Arabidopsis Thailand genome information database (MATDB:

http://mips.gsf.de/proj/thal/db/index.html), oligonucleotide primers for amplifying a DNA sequence having a mini PPR protein gene composed of the two 5th and 6th PPR motifs were prepared (HCF/P5-F and HCF/P6-R; set forth in SEQ ID NOs. 1 and 2). Spe I and Sal I sequences were respectively added onto the 5′ side of the oligonucleotide primers, forward and reverse primers. Spe I and Sal I sequences were integrated so that they can be utilized for cleaving out the inserted sequence with restriction enzyme treatment from the clones obtained.

The DNA fragments comprising the mini PPR protein gene composed of the 5th and 6th PPR motifs were each amplified by performing PCR with 50 μl of reaction solution comprising 100 ng of genome DNA and the above primers in 25 cycles of 95° C. for 30 seconds, 60° C. for 30 seconds, and 72° C. for 30 seconds, and employing KOD-FX (from TOYOBO) as the DNA elongation enzyme. The DNA fragments obtained were cloned with pBAD/Thio-TOPO vector (from Invitrogen) according to the protocol attached to the product. The DNA sequence encoding the cloned mini PPR protein was determined, and this was confirmed to be the sequence homologous to the DNA sequence corresponding to the target in the above database (SEQ ID NO. 79) and designated pHCF152/5&6.

### (Preparation of Mini PPR Protein HCF152/5&6)

The plasmid obtained above was transformed into Escherichia coli TOP10 strain (from Invitrogen). This E. coli was cultured in 300 ml of LB medium where ampicillin was present at a concentration of 100 μg/ml (1 L Erlenmeyer flask comprising 300 mL of medium) at 37° C. When the turbidity of the culture medium reached an absorbance of 0.5 at a wavelength of 600 nm, an inducer L-arabinose was added so that the final concentration was 0.2%, and further cultured for 4 hours. After bacteria collection by centrifugation, bacteria was suspended in 200 ml of buffer A (50 mM Tris-hydrochloric acid, pH 8.0, 500 mM KCl, 2 mM imidazole, 10 mM MgCl2, 0.5% Triton X100, 10% glycerol) comprising 1 mg/ml of lysozyme, and bacteria was destroyed by sonication and freeze-thawing. After centrifugation at 15,000×g for 20 minutes, the supernatant was collected as a crude extraction solution. This crude extraction solution was subjected to a column packed with a nickel column resin (ProBond A, from Invitrogen) equilibrated with buffer A.

Column chromatography was performed by sufficiently washing with buffer A comprising 20 mM imidazole, and then a two-step concentration gradient that elutes the protein of interest with buffer A comprising 200 mM imidazole. The recombinant protein obtained was verified by SDS polyacrylamide gel electrophoresis and detected as a protein of about 30 kDa. This was designated HCF152/5&6 protein. Note that this protein is a fusion protein that has the amino acid sequence set forth in SEQ ID NO. 78, as well as the amino acid sequence of thioredoxin for increasing solubility at the N-terminal side and a histidine tag sequence at the C-terminal side. One hundred microliters of the purified fraction comprising the T-DYW protein was dialyzed with 500 mL of buffer E (20 mM Tris-hydrochloric acid, pH 7.9, 60 mM KCl, 12.5 mM MgCl2, 0.1 mM EDTA, 17% glycerol, and 2 mM DTT) to obtain a purified sample.

### (Preparation of Various Mini PPR Proteins)

Similarly to the above method, gene cloning was performed with mini PPR protein genes composed of two different PPR motifs derived from the HCF152 protein:
for pHCF152/6&7 (SEQ ID NO. 81), primers HCF/P6-F and HCF/P7-R (set forth in SEQ ID NOs. 3 and 4) were employed,
for pHCF152/7&8 (SEQ ID NO. 83), primers HCF/P7-F and HCF/P8-R (set forth in SEQ ID NOs. 5 and 6), were employed
for pHCF152/8&9 (SEQ ID NO. 85), primers HCF/P8-F and HCF/P9-R (set forth in SEQ ID NOs. 7 and 8), were employed
for pHCF152/9&10 (SEQ ID NO. 87), primers HCF/P9-F and HCF/P10-R (set forth in SEQ ID NOs. 9 and 10), were employed
for pHCF152/10&11 (SEQ ID NO. 89), primers HCF/P10-F and HCFP11-R (set forth in SEQ ID NOs. 11 and 12) were employed.

Proteins were similarly prepared, and each was designated HCF152/6&7 (SEQ ID NO. 80), HCF152/7&8 (SEQ ID NO. 82), HCF152/8&9 (SEQ ID NO. 84), HCF152/9&10 (SEQ ID NO. 86), and HCF152/10&11 (SEQ ID NO. 88) proteins (FIG. 1A).

(Preparation of Mini PPR Proteins with Amino Acid Substitution)

Gene cloning was performed with mini PPR proteins with amino acid substitution:
for p5&6/5-T4E (SEQ ID NO. 95), primers HCF5(4T-E)2-F and HCF/P6-R (SEQ ID NOs. 02 and 17) were employed,
for p5&6/5-T4N (SEQ ID NO. 97), primers HCF5(4T-N)2-F and HCF/P6-R (SEQ ID NOs. 02 and 17) were employed,
for p5&6/5-T51 (SEQ ID NO. 99), primers HCF5(T5I)-F and HCF/P6-R (SEQ ID NOs. 02 and 17) were employed,
for p6&7/6-V1R (SEQ ID NO. 139), primers HCF6&7/6#V1R-F and HCF/P7-R (SEQ ID NOs. 04 and 58) were employed.

Proteins were similarly prepared, and each was designated 5&6/5-T4E (SEQ ID NO. 94), 5&6/5-T4N (SEQ ID NO. 96), 5&6/5-T5I (SEQ ID NO. 98), and 6&7/6-V1R proteins (SEQ ID NO. 138).

For p5&6/5-R1A (SEQ ID NO. 91), gene cloning was performed with primers HCF/5&6#R1A-F (SEQ ID NO. 13) and HCF/5&6#R1A-R (SEQ ID NO. 14) as well as pHCF152/5&6 (SEQ ID NO. 79) as the template DNA by site directed mutagenesis kit (from Stratagene) according to the attached protocol. The protein was similarly prepared and designated 5&6/5-R1A (SEQ ID NO. 90) protein.

Similarly to 5&6/5-R1A, gene cloning was performed by site directed mutagenesis kit (from Stratagene) with the following:
for p5&6/5-R1I (SEQ ID NO. 93), primers HCF/5&6#R1I-F and HCF/5&6#R1I-R (SEQ ID NOs. 15 and 16), were employed
for p5&6/5-K8N (SEQ ID NO. 101), primers 5&6/5#K8N-F and 5&6/5#K8N-R (SEQ ID NOs. 20 and 21), were employed
for p5&6/5-K8A (SEQ ID NO. 103), primers 5&6/5#K8A-F and 5&6/5#K8A-R (SEQ ID NOs. 22 and 23), were employed
for p5&6/5-G9L (SEQ ID NO. 105), primers HCF/5&6#G9L-F and HCF/5&6#G9L-R (SEQ ID NOs. 24 and 25), were employed
for p5&6/5-G9A (SEQ ID NO. 107), primers HCF/5&6#G9A-F and HCF/5&6#G9A-R (SEQ ID NOs. 26 and 27), were employed
for p5&6/5-M11A (SEQ ID NO. 109), primers HCF5(M11A)-F and HCF5(M11A)-R (SEQ ID NOs. 28 and 29), were employed
for p5&6/5-M11I (SEQ ID NO. 111), primers HCF5(M11I)-F and HCF5(M11I)-R (SEQ ID NOs. 30 and 31), were employed
for p5&6/5-K12A (SEQ ID NO. 113), primers 5&6/5#K12A-F and 5&6/5#K12A-R (SEQ ID NOs. 32 and 33), were employed
for p5&6/5-K12H (SEQ ID NO. 115), primers HCF5(12K-H)-F and HCF5(12K-H)-R (SEQ ID NOs. 34 and 35), were employed
for p5&6/5-K12N (SEQ ID NO. 117), primers 5&6/5#K12N-F and 5&6/5#K12N-R (SEQ ID NOs. 36 and 37), were employed
for p5&6/5-N13A (SEQ ID NO. 119), primers HCF/5&6#N13A-F and HCF/5&6#N13A-R (SEQ ID NOs. 38 and 39), were employed
for p5&6/5-N13L (SEQ ID NO. 121), primers HCF/5&6#N13L-F and HCF/5&6#N13L-R (SEQ ID NOs. 40 and 41), were employed
for p5&6/5-G14A (SEQ ID NO. 123), primers HCF/5&6#G14A-F and HCF/5&6#G14A-R (SEQ ID NOs. 42 and 43), were employed
for p5&6/5-G14D (SEQ ID NO. 125), primers HCF/5&6#G14D-F and HCF/5&6#G14D-R (SEQ ID NOs. 44 and 45), were employed
for p5&6/6-T4N (SEQ ID NO. 127), primers 5&6/6#T4N-F and 5&6/6#T4N-R (SEQ ID NOs. 46 and 47), were employed
for p5&6/6-T4A (SEQ ID NO. 129), primers 5&6/6#T4A-F and 5&6/6#T4A-R (SEQ ID NOs. 48 and 49), were employed
for p5&6/6-S8A (SEQ ID NO. 131), primers 5&6/6#S8A-F and 5&6/6#S8A-R (SEQ ID NOs. 50 and 51), were employed
for p5&6/6-S8K (SEQ ID NO. 133), primers 5&6/6#S8K-F and 5&6/6#S8K-R (SEQ ID NOs. 52 and 53), were employed
for p5&6/6-N12A (SEQ ID NO. 135), primers 5&6/6#N12A-F and 5&6/6#N12A-R (SEQ ID NOs. 54 and 55), were employed
for p5&6/6-N12R (SEQ ID NO. 137), primers 5&6/6#N12R-F and 5&6/6#N12R-R (SEQ ID NOs. 56 and 57) were employed.

Proteins were similarly prepared, and designated 5&6/5-R1I (SEQ ID NO. 92), 5&6/5-K8N (SEQ ID NO. 100), 5&6/5-K8A (SEQ ID NO. 102), 5&6/5-G9L (SEQ ID NO. 104), 5&6/5-G9A (SEQ ID NO. 106), 5&6/5-M11A (SEQ ID NO. 108), 5&6/5-M11I (SEQ ID NO. 110), 5&6/5-K12A (SEQ ID NO. 112), 5&6/5-K12H (SEQ ID NO. 114), 5&6/5-K12N (SEQ ID NO. 116), 5&6/5-N13A (SEQ ID NO. 118), 5&6/5-N13L (SEQ ID NO. 120), 5&6/5-G14A (SEQ ID NO. 122), 5&6/5-G14D (SEQ ID NO. 124), 5&6/6-T4N (SEQ ID NO. 126), 5&6/6-T4A (SEQ ID NO. 128), 5&6/6-S8A (SEQ ID NO. 130), 5&6/6-S8K (SEQ ID NO. 132), 5&6/6-N12A (SEQ ID NO. 134), and 5&6/6-N12R (SEQ ID NO. 136) proteins.

Moreover, the following were employed for gene cloning with site directed mutagenesis kit (from Stratagene) and pHCF152/6&7 as the template:
for p6&7/7-N4T (SEQ ID NO. 141), primers 6&7#7/N4T-F and 6&7#7/N4T-R (SEQ ID NOs. 59 and 60) were employed,
for p6&7/6-S8K (SEQ ID NO. 143), primers 6&7#6/S8K-F and 6&7#6/S8K-R (SEQ ID NOs. 61 and 62) were employed,
for p6&7/6-S8D (SEQ ID NO. 145), primers 6&7#6/S8D-F and 6&7#6/S8D-R (SEQ ID NOs. 63 and 64) were employed.

Proteins were similarly prepared, and designated 6&7/7-N4T (SEQ ID NO. 140), 6&7/6-S8K (SEQ ID NO. 142), and 6&7/6-S8D (SEQ ID NO. 144) proteins.

Further, the following were employed for gene cloning:
for p8&9/8-D8K (SEQ ID NO. 151), primers 8&9#8/D8K-F and 8&9#8/D8K-R (SEQ ID NOs. 65 and 66) and pHCF152/8&9 as the template were employed;
for p8&9/9-K8D (SEQ ID NO. 153), primers 8&9#9/K8D-F and 8&9#9/K8D-R (SEQ ID NOs. 67 and 68) and pHCF152/8&9 as the template were employed;
for p8&9/8-D8K,9-K8D (SEQ ID NO. 155), primers 8&9#8/D8K-F and 8&9#8/D8K-R (SEQ ID NOs. 65 and 66) and 8&9/9-K8D as the template were employed;
for p5&6/5-K12N,6/N12K (SEQ ID NO. 147), primers 5&6#6/N12K-F and 5&6#6/N12K-R (SEQ ID NOs. 69 and 70) and p5&6#5/K12N as the template were employed;
for p5&6/5-K12M,6/N12R (SEQ ID NO. 149), primers 5&6#5/K12M-F and 5&6#5/K12M-R (SEQ ID NOs. 71 and 72) and p5&6#6N12R as the template were employed.

Proteins were similarly prepared, and designated 8&9/8-D8K (SEQ ID NO. 150), 8&9/9-K8D (SEQ ID NO. 152), 8&9/8-D8K,9-K8D (SEQ ID NO. 154), 5&6/5-K12N,6/N12K (SEQ ID NO. 146), and 5&6/5-K12M, 6/N12R (SEQ ID NO. 148) proteins.

### (Preparation of Substrate RNA)

As the substrate RNA, a 120 base RNA comprising the initiation codon of Arabidopsis Thailand chloroplast petB gene comprising the target sequence endogenous to the at3g09650 protein was employed (see Literature 2). The substrate RNA was designated BD120 (SEQ ID NO. 77). The DNA fragment for synthesizing the substrate RNA BD120 was amplified by performing PCR with oligonucleotide primers BD120-F and BD120-R (SEQ ID NOs. 73 and 74) and 50 μl of reaction solution comprising 10 ng of the above Arabidopsis Thailand genome DNA as the template DNA in 25 cycles of 95° C. for 30 seconds, 60° C. for 30 seconds, and 72° C. for 30 seconds, and employing KOD FX (from TOYOBO) as the DNA elongation enzyme. A T7 promoter sequence for synthesizing the substrate RNA inside a test tube was added to the 5′ terminal side of the BD120-F primer. The DNA fragment obtained was purified by developing in an agarose gel and then cutting out from the gel. With the purified DNA fragment as the template, reaction at 37° C. for 60 minutes in 20 μl of reaction solution comprising NTP mix (10 nmol GTP, CTP, ATP, and 0.5 nmol UTP), 4 μl of [32P] α-UTP (from GE Healthcare, 3000 Ci/mmol), and T7 RNA polymerase (TAKARA BIO INC.) was performed to synthesize the substrate RNA. The substrate RNA was extracted with phenol/chloroform, precipitated in ethanol, then the full amount was developed in a denaturing 6% polyacrylamide gel electrophoresis comprising 6 M urea, and exposed to an X-ray film for 60 seconds to detect the 32P-labeled RNA. The 32P-labeled RNA was cut out from the gel, immersed in 200 μl of gel eluate (0.3 M sodium acetate, 2.5 mM EDTA, and 0.01% SDS) at 4° C. for 12 hours to elute the RNA from the gel. The radioactivity of 1 μl of the eluted RNA was measured to calculate the total amount of the RNA synthesized. After ethanol precipitation, RNA was dissolved in ultrapure water to make 2500 cpm/μl (1 fmol/μl). This preparation method ordinarily yields about 100 μl of 2500 cpm/μl RNA.

### (RNA Binding Ability of Mini PPR Protein)

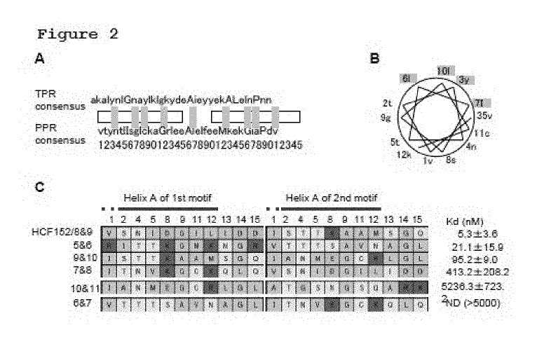

The RNA binding activity of the mini PPR protein was analyzed by gel shift method. To 20 μl of reaction solution (10 mM Tris-hydrochloric acid, pH 7.9, 30 mM KCl, 6 mM MgCl2, 2 mM DTT, 8% glycerol, and 0.0067% Triton X-100), 375 pM (7. 5 fmol/20 μL) of the above substrate RNA (BD120) and 0-3750 nM of mini PPR protein was mixed, and this was reacted at 25° C. for 15 minutes. Then, to the reaction solution was added 4 microliters of 80% glycerol solution, and 10 μL was developed in 10% nondenaturing polyacrylamide gel comprising 1×TBE (89 mM Tris-HCl, 89 mM Boric acid, and 2 mM EDTA) and the gel was dried after electrophoresis. The radioactivity of RNA in the gel was measured with Bioimaging Analyzer BAS2000 (From Fujifilm). The results are shown in FIGS. 1C-H. As shown in FIGS. 1C-H, the binding of protein and RNA is manifested as the difference in mobility of the 32P-labeled RNA. This is because the molecular weight of the 32P-labeled RNA/protein conjugate is larger than the molecular weight of the 32P-labeled RNA alone and thus mobility in electrophoresis had become slower. The binding between protein and RNA was quantified based on the results in FIGS. 1C-H (FIG. 1B), and evaluated by determining the disassociation constant (Kd) (FIG. 2C). ND (not determined) was assigned when Kd could not be calculated even when 3750 nM of protein was employed.

As shown in FIG. 2C, it became clear that each mini PPR protein expresses a different RNA affinity. For example, the RNA affinity of HCF152/8&9 and HCF152/10&11 proteins is Kd=5.3 nM and Kd=5236.3 nM, respectively, and the difference in RNA affinity thereof is more than 1000 folds.

Next, amino acids responsible for the difference in RNA affinity described above were predicted. As shown above, it is predicted from the sequence information that the PPR motif is composed of two α helices and is classified as a helical repeat protein family in the broad sense (FIG. 2A; Non-Patent Literature 4 above). This family includes the puf motif configuring the aforementioned pumilio protein (36 amino acids; three helices), as well as TPR (34 amino acids; two helices), ARM (38 amino acids; three helices), and HEAT (34 amino acids; two helices) etc., and all alike show a general structure of a semi-donut or crescent shape (see Literature 3).

From the amino acid sequence of the PPR motif and preliminary structural prediction, it was predicted that amino acids that act on RNA binding are allocated in Helix A. Accordingly, focus was placed on amino acids contained in Helix A. In the PPR motif, Helix A is composed of the 2nd-12th amino acids. The 1st amino acid may or may not be contained in the helix (shown with dotted line; FIG. 2C). Comparing the conserved sequence of the TPR motif (acts on protein/protein interaction) that is very similar to the PPR motif, it was predicted that the amino acids shown in gray in FIGS. 2A and B form the skeleton of the α helix (the 3rd, 6th, 7th, and 10th amino acids of Helix A of the PPR motif), and as shown in FIG. 2B, it was found that they are concentrated at one site when the helix is seen from the side. It is a known fact that the α helix completes a rotation in 3.6 amino acids and is a dextral structure of 5.4 A units.

### (Characterization of 8th Amino Acid)

In FIG. 2C, mini PPR proteins were aligned in the order from the highest affinity with RNA (lowest Kd), and among the amino acids contained in Helix A, those other than the amino acids shown in gray above were shown. Accordingly, with the exception of HCF15 2/5&6, it was found that in mini PPR proteins composed of two PPR motifs that have high affinity with RNA, a basic amino acid (K and R; lysine and arginine) and an acidic amino acid (D and E; aspartic acid and glutamic acid) appear as a pair in the 8th amino acid of the first PPR motif and the 8th amino acid of the second PPR motif (in no particular order).

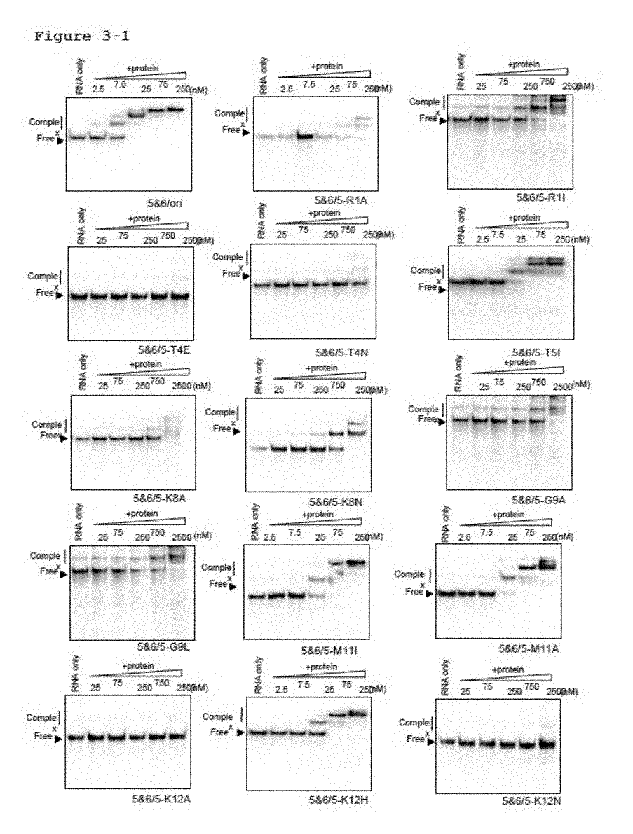

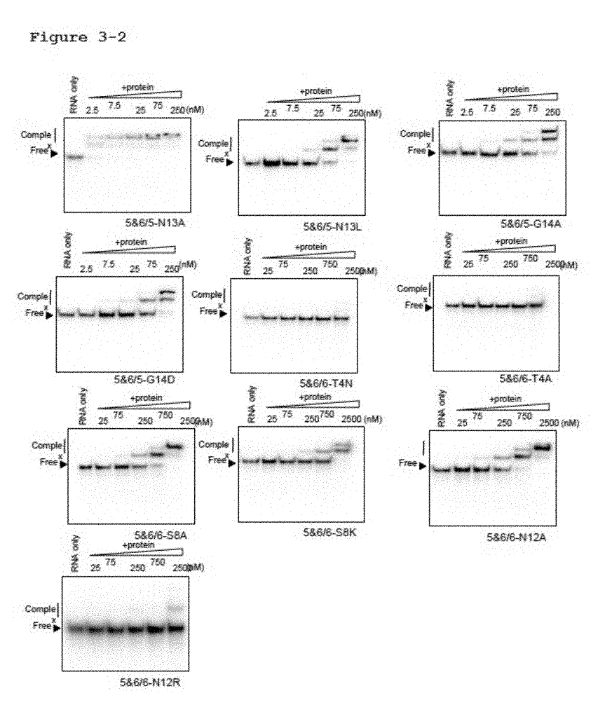

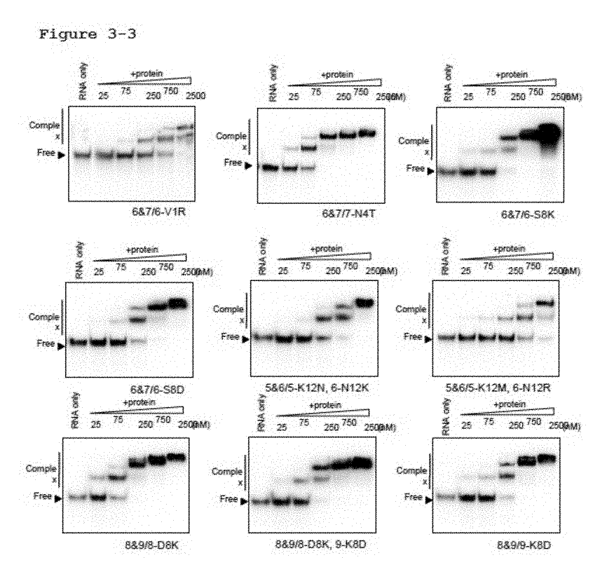

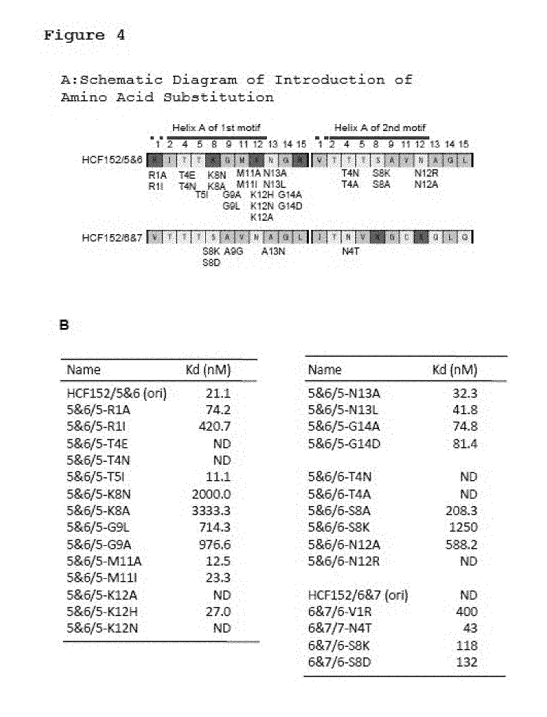

Accordingly, the 8th amino acid serine (S) of the first PPR motif of HCF152/6&7 mini PPR protein which had an affinity with RNA below the detection limit (ND; Kd>3750 nM) was substituted to aspartic acid (D) to prepare 6&7/6-S8D. The RNA binding ability was determined, and significant improvement of RNA affinity (Kd=200) was observed (FIGS. 3-3 and 4B). This shows that the RNA affinity of the mini PPR protein can be improved by at least about 20 folds by substituting the 8th amino acid to aspartic acid, in other words that the 8th aspartic acid acts actively on RNA binding (described below in detail).

(Identification of Amino Acids that Act on RNA Binding)

It was anticipated that amino acids that act on RNA binding were also allocated near the 8th position on the helical structure. Accordingly, based on the amino acid allocation shown in FIG. 2B, focus was placed on the 2nd-11th (1st, 2nd, 4th, 5th, 8th, 9th, 11th, and 12th) amino acids located in the left bottom half of the circular helix. Using HCF152/5&6 as the model, mini PPR proteins having one amino acid substitution introduced centering on the aforementioned positions (1st, 2nd, 4th, 5th, 8th, 9th, 11th, and 12th) were prepared. The amino acid substitutions were based on substitution to alanine. However, since there are positions in the PPR motif that contain alanine, the effect from amino acid substitution was verified by substituting the same position to another different amino acid (FIG. 4A). Affinity with RNA was analyzed by the same gel shift method as above (FIG. 3), and the affinity with the RNA was evaluated with Kd (FIG. 4B).

As shown in FIG. 4B, mini PPR proteins with amino acid substitution introduced showed various Kd (affinity with RNA), and there were cases where RNA affinity was reduced (Kd was elevated) by amino acid substitution and where almost no effect by amino acid substitution was seen. In this analysis, a protein in which RNA affinity was significantly elevated (Kd was reduced) was not obtained. Since the Kd of a natural mini PPR protein HCF152/5&6 is 21.1 nM, by defining a reduction of RNA affinity by 10 folds or more as significant reduction of RNA affinity by amino acid substitution, it was evaluated that introduction of substitution into the five amino acids of 1st, 4th, 8th, 9th, and 12th amino acids (numbering is the amino acid number configuring the PPR motif configuration) significantly reduced RNA affinity. In other words, this means that the RNA affinity of the PPR protein can be reduced by substituting the five 1st, 4th, 8th, 9th, and 12th amino acids to a different amino acid.

(Identification of Amino Acids that Act Actively on RNA Binding)

Subsequently, in order to evaluate the analysis by the amino acid substitution above in more detail, it was investigated whether elevation of RNA affinity is observed by substituting the 1st, 4th, and 8th amino acids of HCF152/6&7 mini PPR protein in which affinity with RNA was below the detection limit (ND; Kd>3750 nM) to an amino acid possessed by a mini PPR protein with high RNA affinity (such as HCF152/8&9). Amino acid substitution was not introduced for the 9th and 12th amino acids in this analysis because amino acids unique only to HCF152/6&7 could not be found (described below).

As a result, improvement of RNA affinity was observed by substituting the 1st valine (V) of the first PPR motif with arginine (R), the 4th asparagine (N) of the second PPR motif with threonine (T), and the 8th serine (S) of the first PPR motif with lysine (K) or aspartic acid (D) (FIG. 3-3). In other words, it means that by allowing the 1st, 4th, and 8th amino acids to act actively on RNA affinity and manipulating the 1st, 4th, and 8th amino acids, the RNA affinity of the PPR motif, and in turn the PPR protein can be improved.

### (Characterization of 12th Amino Acid)

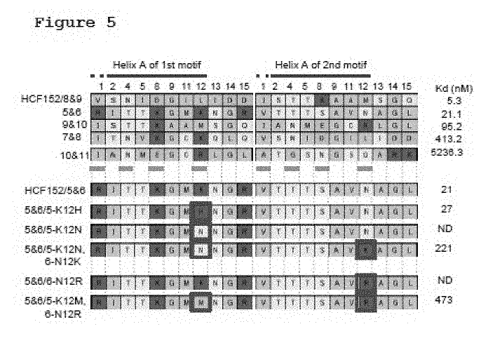

Looking at the composition of the 12th amino acid of mini PPR proteins, in many cases it is basic in one motif and neutral or hydrophobic in the other motif. Accordingly, the significance of this combination of basic and neutral (hydrophobic) was verified. Using HCF152/5&6, when the 12th lysine (K) of the first PPR motif was substituted to a similarly basic histidine (H), RNA affinity (Kd) almost equivalent to that in nature was shown (5&6/5-K12H; FIGS. 3-1 and FIG. 5). However, significant reduction of RNA affinity was observed (5&6/5-K12N; Kd=ND (>3750 nM)) when the same amino acid was substituted to asparagine (N). However, when the 12th amino acid of the second PPR motif which is asparagine (N) in this amino acid substituted protein was substituted to a basic amino acid lysine (K), RNA affinity improved (5&6/5-K12N,6-N12K; FIGS. 3-3 and 5). In other words, reduction of RNA affinity with substitution of the 12th amino acid of the first motif (K->N) is complemented by substitution of the 12th amino acid of the second motif (N→>K).

Since simple improvement of RNA affinity by allocation of the 12th amino acid to a basic amino acid (improvement in affinity with acidic RNA) was conceived, the 12th amino acid asparagine of the second motif was subsequently substituted to arginine (R), and significant reduction of RNA affinity was also observed in this case (5&6/6-N12R; Kd=ND). Accordingly, similarly to the above, by keeping the arginine substitution and substituting the 12th lysine (K) of the first motif to a hydrophobic amino acid methionine (M), a slight improvement of RNA affinity was similarly observed (5&6/5-K12M,6-N12R; Kd=473 nM; FIG. 5).

From this analysis, this means that it is important that the 12th amino acid also acts actively on the RNA affinity of the PPR motif, and that the 12th amino acids in the two motifs are a pair of basic and neutral (or hydrophobic) amino acids.

Moreover, this also means that the PPR motif does not act alone, but the RNA binding property of the whole is regulated by the balance with the amino acids contained in the previous and next motifs. This means that improvement of RNA affinity can be achieved by making the 12th amino acids a pair of basic and neutral (hydrophobic) when designing the RNA binding factor with a combination of multiple PPR motifs.

### (Characterization of 8th Amino Acid)

In the 12th amino acid, it was found that interaction between adjacent PPR motifs affects RNA affinity. In the mini PPR proteins employed here, the tendency of RNA affinity to be high is observed when the 8th amino acids in two PPR motifs are a pair of basic and acidic, and in fact, it has previously been shown that the 8th amino acid acts actively on RNA affinity (FIGS. 2 and 4).

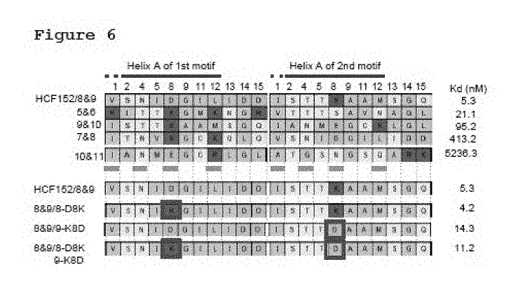

Accordingly, using HCF152/8&9 as the model, characterization of the 8th amino acid was performed. When aspartic acid (D) of the first PPR motif was changed to lysine (K) to obtain a pair of basic and basic, there was no change in RNA affinity (8&9/8-D8K; FIG. 6). Similarly, when lysine (K) of the second PPR motif was changed to aspartic acid (D) to obtain a pair of acidic and acidic (8&9/9-K8D), nor when a pair of basic and acidic was inverted to a pair of acidic and basic (8&9/8-D8K,9-K8D), no significant difference in RNA affinity could be seen.

This means, in contrast to the 12th amino acid, RNA affinity is retained if either one of acidic or basic amino acid is allocated as the 8th amino acid. In other words, this suggests that regulation is possible by improving RNA affinity by making the 8th amino acid a basic or an acidic amino acid, or by reducing RNA affinity by having it otherwise (such as asparagine or alanine).

### Example 2

### Statistical Analysis of Amino Acids Configuring the PPR Motif

In a protein domain search program Pfam on the web (Pfam: http://pfam.sanger.ac.uk/), 558 PPR motif sequences were obtained from PF01535 defined as the PPR motif (SEQ ID NO. 156). From the sequences obtained, the composition of the 1st, 4th, 8th, and 12th amino acid sequences was analyzed. As a result, it became clear that most of the 1st amino acid was composed of hydrophobic amino acids and the 4th of neutral amino acids. The 8th amino acid was most often neutral (43%), but composed of basic, acidic, and hydrophobic amino acids (each about 20%). The 12th amino acid was most often basic amino acids (55%), but also in many cases composed of neutral amino acids (22%). In this way, it was suggested that since the 1st, 4th, 8th, and 12th amino acids differ in their nature, each amino acid plays a different role in the RNA binding ability of the PPR motif.

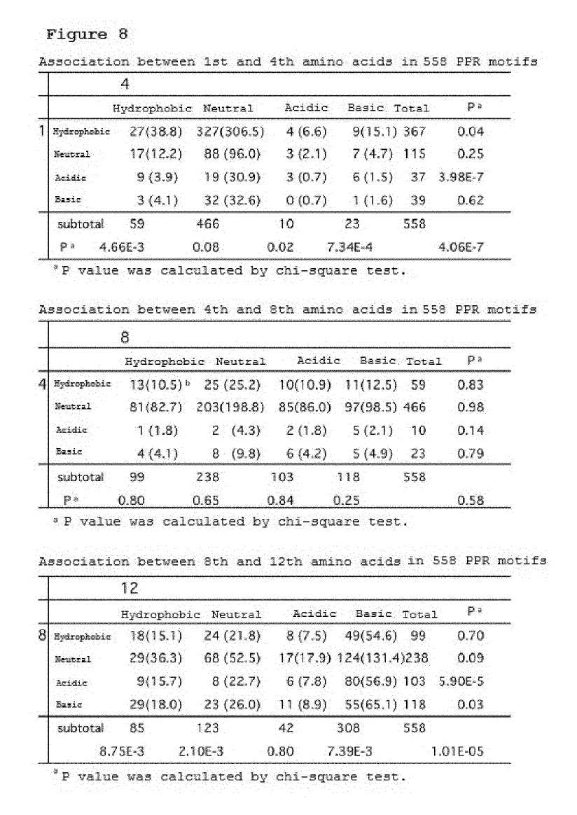

Subsequently, the bias of the combination of amino acids that appear at the 1st and 4th positions on the same motif was analyzed, and the bias with the theoretical value was evaluated by chi-square test (FIG. 8). Similarly, the combination of 4th and 8th amino acids as well as the combination of 8th and 12th amino acids were analyzed. As a result, significant bias became clear in the combination of 1st and 4th as well as 8th and 12th amino acids (P value<0.05; 5% significance level). In other words, it is suggested that 1st and 4th as well as 8th and 12th amino acids cooperate to act on RNA binding. Note that a neutral amino acid in this test is those that are neutral and hydrophilic, i.e. asparagine, serine, glutamine, threonine, tyrosine, and cysteine. A hydrophobic amino acid in this test is tryptophan, glycine, methionine, proline, phenylalanine, alanine, valine, leucine, and isoleucine, as is previously defined herein.

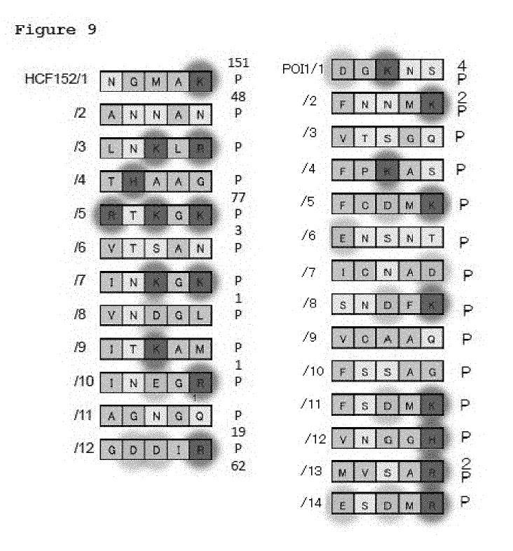

Further, the 1st, 4th, 8th, and 12th amino acids of each motif in a full length PPR protein HCF152 (twelve PPR motifs, SEQ ID NOs. 75 and 76) were analyzed, and it was found that a basic amino acid appears at the 12th position in almost every other motif. The phase of this basic amino acid is composed of two locations which are the 1st to 7th and the 10th to 12th. Similarly in the 8th amino acid, a similar basic amino acid phase in every other motif appears in the 3rd to 9th PPR motifs. On the other hand, it was found that a phase of every other acidic amino acid is present in the 8th to 12th PPR motifs (FIG. 9).

In order to verify the universality of this phase, the 1st, 4th, 8th, and 12th amino acids of each motif in a different PPR protein LOI1 (14 PPR motifs) were analyzed. Consequently, it was found that in the LOI1 protein, a basic amino acid appears in every two of the 12th amino acid of the 2nd to 11st PPR motifs, and a phase of an acidic amino acid in every two of the 8th amino acid in the 5th to 14th PPR motifs appears (FIG. 9). In other words, in a protein composed of multiple PPR motifs, it is suggested that there is a possibility that protein function, i.e. RNA binding activity can be elevated by allocating an acidic or a basic amino acid at the 8th and 12th amino acid in every other or every two motifs.

It is thought that sequence specific RNA binding ability is exerted in a PPR protein when PPR motifs of differing nature are in succession. In the substitution experiment of the 8th and 12th amino acids shown above, binding RNA sequence specificity did not change. The results of the above statistical analysis suggest that the binding RNA specificity of the PPR motif is determined centering on the 1st and 4th amino acids, and that there is a possibility that binding RNA sequence specificity can be altered by altering those amino acids. However, the possibility of altering the binding RNA sequence specificity by substituting the 8th and 12th amino acid is not to be denied.

### Example 3

### (Preparation of Substrate RNA)

As the substrate RNA, a 25-base nucleotide homopolymer (LN25) having a linker AUCG added at the 5′ terminal side was chemically synthesized (LA25, SEQ ID NO. 157; LU25, SEQ ID NO. 158; LG25, SEQ ID NO. 159; and LC25, SEQ ID NO. 160; consigned to Thermo SCIENTIFIC). A ³²P label was added to the 5′ terminal of the synthesized RNA with T4 polynucleotide kinase (from Takara) and γ[³²P] ATP (from MP Biomedical, 6000 Ci/mmol). After ethanol precipitation, the labeled RNA was dissolved to 5 fmol/μL to prepare radioactively labeled RNA.

### (Preparation of Mini PPR Protein)

Similarly to the method described above, recombinant protein expression vectors and proteins were prepared:
for pHCF152/2&3 (SEQ ID NO. 169), primers HCF/P2-F and HCF/P3-R (set forth in SEQ ID NOs. 161 and 162) were employed,
for pHCF152/3&4 (SEQ ID NO. 171), primers HCF/P3-F and HCF/P4-R (set forth in SEQ ID NOs. 163 and 164) were employed,
for pCRR4/6 (SEQ ID NO. 173), primers CAR4/6-F and CAR4/6-R (set forth in SEQ ID NOs. 165 and 166) were employed,
for pCRR4/6(I1L) (SEQ ID NO. 175), primers CAR4/6 (I1L)-F and CAR4/6-R (set forth in SEQ ID NOs. 167 and 166) were employed, and each was designated HCF152/2&3 (SEQ ID NO. 168), HCF152/3&4 (SEQ ID NO. 170), CAR4/6 (SEQ ID NO. 172), and CAR4/6 (I1L) (SEQ ID NO. 174) proteins.

### (Binding Specificity of Mini PPR Protein)

The binding specificity of the mini PPR proteins were analyzed by gel shift method. To 20 μl of reaction solution (10 mM Tris-hydrochloric acid, pH 7.9, 30 mM KCl, 6 mM MgCl₂, 2 mM DTT, 8% glycerol, 0.0067% Triton X-100), 4 pM (5 fmol/20 μL) of the above substrate RNA (LN25; LA25, LU25, LG25, or LC25) and 200 nM of mini PPR protein was mixed, and this was reacted at 25° C. for 15 minutes. Then, to the reaction solution was added 4 μl of 80% glycerol solution, and 10 μL was developed in 10% nondenaturing polyacrylamide gel comprising 1×TBE (89 mM Tris-HCl, 89 mM Boric acid, and 2 mM EDTA) and the gel was dried after electrophoresis. The radioactivity of RNA in the gel was measured with Bioimaging Analyzer BAS2000 (From Fujifilm).

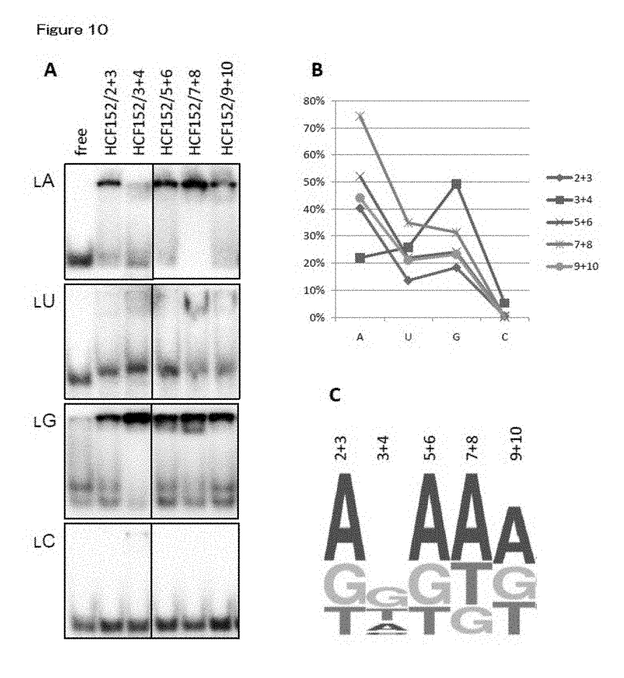

The results are shown in FIG. 10. As shown in FIG. 10A, the binding of protein and RNA is manifested as the difference in mobility of the ³²P-labeled RNA. This is because the molecular weight of the ³²P-labeled RNA/protein conjugate is larger than the molecular weight of the ³²P-labeled RNA alone and thus mobility in electrophoresis had become slower. The binding of each mini PPR protein with each of A, U, G, and C was quantified based on radioactivity (FIG. 10B), and visualized with WebLOGO (HYPERLINK “http://weblogo.berkeley.edu/”http://weblogo.berkeley.edu/) (FIG. 10C). As shown in FIG. 10C, the binding base specificity of each mini PPR protein was A>G>U for HCF152/2&3, 5&6, and 9&10, particularly binding strongly to A. HCF152/7&8 also binds strongly to A, but the binding base specificity thereof was A>U>G. In the meantime, HCF152/3&4 bound well with G, and the binding base specificity thereof was G>U>A.

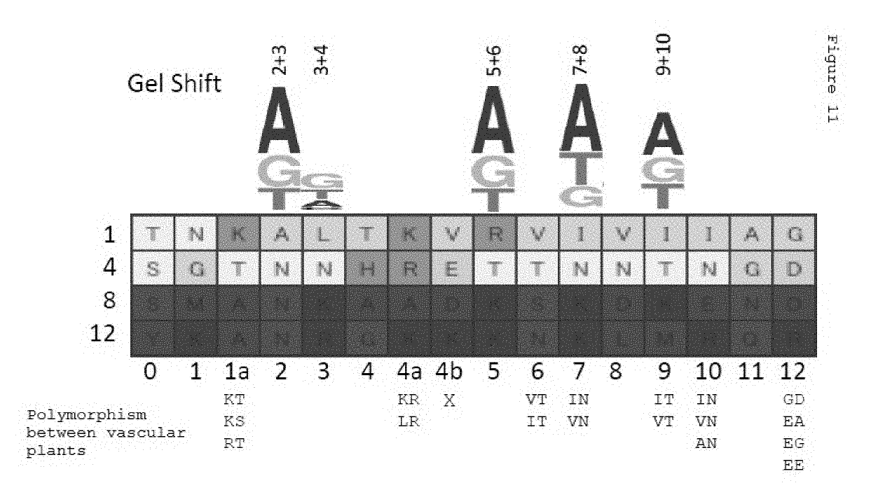

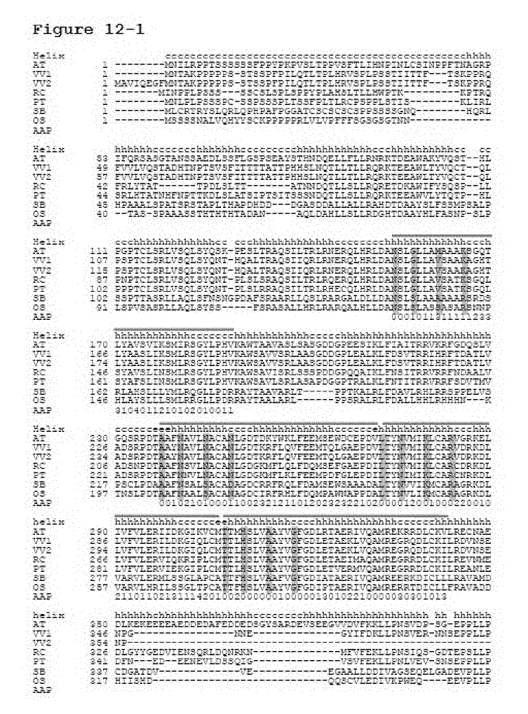

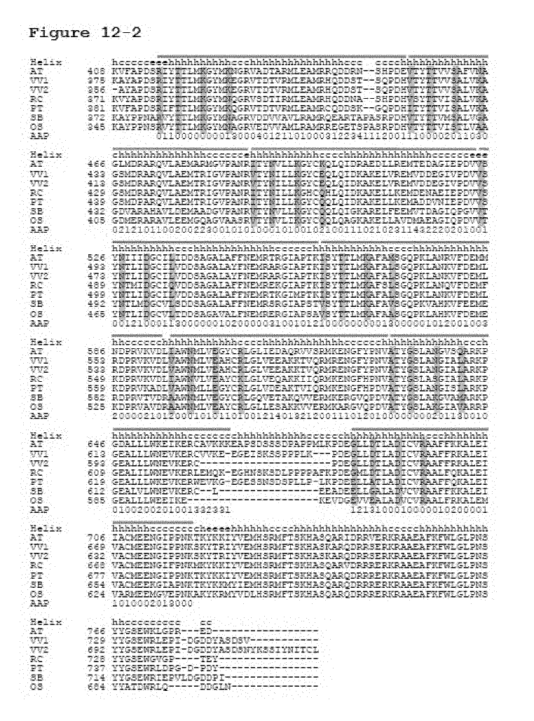

As described above, it was thought that the binding base specificity of a PPR motif configuring a mini PPR protein is determined by the 1st and 4th amino acids in the motif. Accordingly, focus was placed on the 1st and 4th amino acids in each mini PPR protein (FIG. 11). Because the 1st and 4th amino acids in each PPR motif were diverse, potential homologous proteins of Arabidopsis Thailand HCF152 protein were searched in NCBI BLAST from 6 species of vascular plants to analyze the polymorphism of the aforementioned amino acids (FIG. 12). As a result, when the 1st amino acid was isoleucine and the 4th amino acid was asparagine (IN) (the seventh and tenth PPR motif), polymorphism of valine (V) and alanine (A) was seen in the 1st amino acid; and when the 1st amino acid was valine and the 4th amino acid was threonine (VT) (the sixth PPR motif), polymorphism of isoleucine (I) was seen in the first amino acid (FIG. 12). In other words, it is suggested that these polymorphic amino acids are amino acids that are responsible for the same function.

Combining these results, when the 1st and 4th amino acids are valine and threonine (VT) or isoleucine and threonine (IT), the motif has binding sequence specificity that binds strongly to A, then to U, and then to G; when the 1st and 4th amino acids are valine and asparagine (VN) or isoleucine and asparagine (IN) or alanine and asparagine (AN), the motif has specificity that binds strongly to A, then to G, and then to U; and when the 1st and 4th amino acids are leucine and asparagine (LN), the motif has specificity that binds strongly to G, then to T, and then to A.

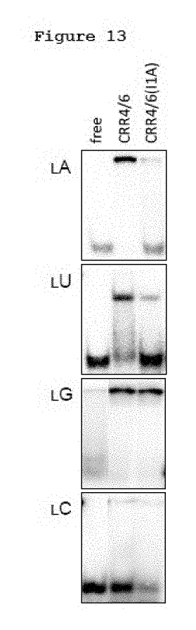

The mini PPR proteins employed in the experiments are composed of two PPR motifs, but it was thought that it is the nature of the first PPR motif is largely expressed. In order to investigate the roles of the 1st and 4th amino acids in the PPR motif in detail, a protein composed of one PPR motif was prepared with a different PPR protein CAR4 (at2g45350, SEQ ID NOs. 176 and 177) and analyzed. As shown in FIG. 13, protein CAR4/6 having isoleucine and asparagine (I and N) as the 1st and 4th amino acids has a preference to bind to A, but protein CAR4/6 (I1A) where the 1st amino acid is altered to leucine and having leucine and asparagine (I and N) as the 1st and 4th amino acids did not bind to A but bound well to G.

In other words, this means that by employing a PPR motif corresponding to the RNA recognition code for each of the 1st and 4th amino acids, a protein that binds to each of the bases can be prepared, and moreover, construction of a protein that binds to a RNA sequence having consecutive aforementioned bases is possible by linking.

### REFERENCES CITED IN THE EXAMPLES

Reference 1 : Meierhoff, K., Felder, S., Nakamura, T., Bechtold, N., and Schuster, G. (2003). HCF152, an Arabidopsis

RNA binding pentatricopeptide repeat protein involved in the processing of chloroplast psbB-psbT-psbH-petB-petD RNAs. Plant Cell 15, 1480-1495.

Reference 2: Nakamura, T., Meierhoff, K., Westhoff, P., and Schuster, G. (2003).

RNA-binding properties of HCF152, an Arabidopsis PPR protein involved in the processing of chloroplast RNA. Eur. J. Biochem. 270, 4070-4081.

Reference 3 :Edwards, T. A., Pyle, S. F., Wharton, R. P., and Aggarwal, A. K. (2001). Structure of Pumilio reveals similarity between RNA and peptide binding motifs. Cell 105, 281-289.

1. A method for altering the RNA binding property of a PPR protein having one or more (preferably 2 or more, more preferably 2-14) PPR motifs that consist of a polypeptide with a length of 30-38 amino acids represented by Formula I:
[Chemical Formula 1]
-Xᵢ-(Helix A)-Xᵢᵢ-(Helix B)-Xᵢᵢᵢ- (Formula I)
(wherein:
Helix A is a portion with a length of 12 amino acids that can form an a helix structure, Helix A is represented by Formula II:
[Chemical Formula 2]
-A₁-A₂-A₃-A₄-A₅-A₆-A₇-A₈-A₉-A₁₀-A₁₁-A₁₂- (Formula II)
(A₁-A₁₂ each independently represent an amino acid);
Helix B is a portion with a length of 11-13 amino acids that can form an a helix structure; and
X,ᵢ₋ᵢᵢᵢ are each independently a portion consisting of a length of 1-9 amino acids or does not exist,)
comprising a step of substituting one or more amino acids selected from the group consisting of A₁, A₄, A₈, A₉, and A₁₂ (preferably the group consisting of A₁, A₄, A₈, and A₁₂) in the one or more PPR motifs with a different amino acid.

2. A method according to claim 1 wherein the method is for improving the RNA binding activity of the PPR protein, the PPR protein has two or more PPR motifs, and the method comprises any of the following steps of:
a substitution to make A₁ of the first PPR motif a basic amino acid, preferably arginine;
a substitution to make A₄ of the second PPR motif a neutral amino acid, preferably threonine;
a substitution to make A₈ of the first PPR motif a basic amino acid, preferably lysine, or an acidic amino acid, preferably aspartic acid; and
a substitution of A₁₂ of the first PPR motif and/or A₁₂ of the second PPR motif to make either one a basic amino acid and the other a neutral amino acid or a hydrophobic amino acid.

3. A method according to claim 1 comprising an alteration that considers the following in the one or more PPR motifs:
cooperation between A₁ of a motif and A₄ of the same motif, and/or
cooperation between A₈ of a motif and A₁₂ of the same motif.

4. A method for designing a protein having RNA binding property that employs a PPR motif according to claim 1 that has a basic or acidic amino acid at A₈ and A₁₂.

5. A method for designing a protein that can specifically bind to a target base in a RNA, comprising employing a PPR motif according to claim 1, wherein A₁ and A₄ is the combination to which the target base is specifically bound to improve the base binding specificity of that motif.

6. A method according to claim 5, wherein the target base is adenine, uracil, or guanine (preferably adenine or uracil, and more preferably adenine), and the combination of A₁ and A₄ is valine and threonine or isoleucine and threonine; or
the target base is adenine, guanine, or uracil (preferably adenine or guanine, and more preferably adenine), and the combination of A₁ and A₄ is valine and asparagine, isoleucine and asparagine, or alanine and asparagine in that order; or
the target base is guanine, thymine, or adenine (preferably guanine or thymine, and more preferably guanine), and the combination of A₁ and A₄ is leucine and asparagine in that order.

7. A method according to claim 5, comprising employing PPR motifs corresponding to each of two or more target bases in a RNA, wherein A₁ and A₄ in one motif is the combination to which the corresponding target base is specifically bound to improve the binding specificity of that motif; and A₁ and A₄ in another motif is the combination to which the corresponding target base is specifically bound to improve the base binding specificity of each motif.

8. A protein comprising all or a portion with RNA binding activity of a polypeptide consisting of an amino acid sequence of SEQ ID NOs. 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 168, 170, 172, or 174.

9. A polynucleotide encoding a RNA binding protein according to claim 8.

10. A polynucleotide according to claim 9 having a base sequence of SEQ ID NOs. 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 169, 171, 173, or 175.

11. A method for regulating RNA function that employs a PPR protein altered with a method according to claim 1, or a protein comprising all or a portion with RNA binding activity of a polypeptide consisting of an amino acid sequence of SEQ ID NOs. 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 168, 170, 172, or 174.

12. A method according to claim 2 comprising an alteration that considers the following in the one or more PPR motifs:
cooperation between A₁ of a motif and A₄ of the same motif, and/or
cooperation between A₈ of a motif and A₁₂ of the same motif.

13. A method according to claim 6, comprising employing PPR motifs corresponding to each of two or more target bases in a RNA, wherein A₁ and A₄ in one motif is the combination to which the corresponding target base is specifically bound to improve the binding specificity of that motif; and A₁ and A₄ in another motif is the combination to which the corresponding target base is specifically bound to improve the base binding specificity of each motif.

14. A method for regulating RNA function that employs a PPR protein altered with a method according to claim 2.

15. A method for regulating RNA function that employs a PPR protein altered with a method according to claim 3.

16. A method for regulating RNA function that employs a PPR protein altered with a protein designed with a method according to claim 4.

17. A method for regulating RNA function that employs a PPR protein altered with a protein designed with a method according to claim 5.

18. A method for regulating RNA function that employs a PPR protein altered with a protein designed with a method according to claim 6.

19. A method for regulating RNA function that employs a PPR protein altered with a protein designed with a method according to claim 7.

20. A method for regulating RNA function that employs a PPR protein altered with a protein according to claim 8.


SEQUENCE LISTING (177 sequences)

SEQ ID NO: 1 - DNA, 34 bp, Artificial Sequence
  Description of Artificial Sequence Synthetic primer
cgcactagtaggatctacacgacgttgatgaaag

SEQ ID NO: 2 - DNA, 33 bp, Artificial Sequence
  Description of Artificial Sequence Synthetic primer
cgcgtcgaccctatttgcaggaacacccatccg

SEQ ID NO: 3 - DNA, 34 bp, Artificial Sequence
  Description of Artificial Sequence Synthetic primer
cgcactagtgttacatacactacggttgtgtcag

SEQ ID NO: 4 - DNA, 33 bp, Artificial Sequence; identical to SEQ ID NO: 2
  Description of Artificial Sequence Synthetic primer

SEQ ID NO: 5 - DNA, 35 bp, Artificial Sequence
  Description of Artificial Sequence Synthetic primer
cgcactagtattacttataatgttctgctcaaagg

SEQ ID NO: 6 - DNA, 32 bp, Artificial Sequence
  Description of Artificial Sequence Synthetic primer
cgcgtcgaccttagttggtgcaatccctctcg

SEQ ID NO: 7 - DNA, 34 bp, Artificial Sequence
  Description of Artificial Sequence Synthetic primer
cgcactagtgtttcctataacattataatagatg

SEQ ID NO: 8 - DNA, 34 bp, Artificial Sequence
  Description of Artificial Sequence Synthetic primer
cgcgtcgacatcaactttgacccttggatcattc

SEQ ID NO: 9 - DNA, 34 bp, Artificial Sequence
  Description of Artificial Sequence Synthetic primer
cgcactagtattagttacacaactttgatgaagg

SEQ ID NO: 10 - DNA, 33 bp, Artificial Sequence
  Description of Artificial Sequence Synthetic primer
cgcgtcgaccacatttgggtaaaacccgttttc

SEQ ID NO: 11 - DNA, 34 bp, Artificial Sequence
  Description of Artificial Sequence Synthetic primer
cgcactagtatcgcgtggaacatgttggttgaag

SEQ ID NO: 12 - DNA, 34 bp, Artificial Sequence
  Description of Artificial Sequence Synthetic primer
cgcgtcgactttctttttcaccgcacacctttcc

SEQ ID NO: 13 - DNA, 44 bp, Artificial Sequence
  Description of Artificial Sequence Synthetic primer
gctcgcccttcgcactagtgcgatctacacgacgttgatgaaag

SEQ ID NO: 14 - DNA, 44 bp, Artificial Sequence
  Description of Artificial Sequence Synthetic primer
ctttcatcaacgtcgtgtagatcgcactagtgcgaagggcgagc

SEQ ID NO: 15 - DNA, 44 bp, Artificial Sequence
  Description of Artificial Sequence Synthetic primer
gctcgcccttcgcactagtattatctacacgacgttgatgaaag

SEQ ID NO: 16 - DNA, 44 bp, Artificial Sequence
  Description of Artificial Sequence Synthetic primer
ctttcatcaacgtcgtgtagataatactagtgcgaagggcgagc

SEQ ID NO: 17 - DNA, 35 bp, Artificial Sequence
  Description of Artificial Sequence Synthetic primer
cgcactagtaggatctacgaaacgttgatgaaagg

SEQ ID NO: 18 - DNA, 35 bp, Artificial Sequence
  Description of Artificial Sequence Synthetic primer
cgcactagtaggatctacaacacgttgatgaaagg

SEQ ID NO: 19 - DNA, 34 bp, Artificial Sequence
  Description of Artificial Sequence Synthetic primer
cgcactagtaggatctacacgattttgatgaaag

SEQ ID NO: 20 - DNA, 43 bp, Artificial Sequence
  Description of Artificial Sequence Synthetic primer
ggatctacacgacgttgatgaatggttatatgaagaatgggcg

SEQ ID NO: 21 - DNA, 43 bp, Artificial Sequence
  Description of Artificial Sequence Synthetic primer
cgcccattcttcatataaccattcatcaacgtcgtgtagatcc

SEQ ID NO: 22 - DNA, 43 bp, Artificial Sequence
  Description of Artificial Sequence Synthetic primer
ggatctacacgacgttgatggcaggttatatgaagaatgggcg

SEQ ID NO: 23 - DNA, 43 bp, Artificial Sequence
  Description of Artificial Sequence Synthetic primer
cgcccattcttcatataacctgccatcaacgtcgtgtagatcc

SEQ ID NO: 24 - DNA, 41 bp, Artificial Sequence
  Description of Artificial Sequence Synthetic primer
ctacacgacgttgatgaaactgtatatgaagaatgggcgtg

SEQ ID NO: 25 - DNA, 41 bp, Artificial Sequence
  Description of Artificial Sequence Synthetic primer
cacgcccattcttcatatacagtttcatcaacgtcgtgtag

SEQ ID NO: 26 - DNA, 41 bp, Artificial Sequence
  Description of Artificial Sequence Synthetic primer
ctacacgacgttgatgaaagcgtatatgaagaatgggcgtg

SEQ ID NO: 27 - DNA, 41 bp, Artificial Sequence
  Description of Artificial Sequence Synthetic primer
cacgcccattcttcatatacgctttcatcaacgtcgtgtag

SEQ ID NO: 28 - DNA, 41 bp, Artificial Sequence
  Description of Artificial Sequence Synthetic primer
gacgttgatgaaaggttatgcgaagaatgggcgtgtggcag

SEQ ID NO: 29 - DNA, 41 bp, Artificial Sequence
  Description of Artificial Sequence Synthetic primer
ctgccacacgcccattcttcgcataacctttcatcaacgtc

SEQ ID NO: 30 - DNA, 41 bp, Artificial Sequence
  Description of Artificial Sequence Synthetic primer
gacgttgatgaaaggttatattaagaatgggcgtgtggcag

SEQ ID NO: 31 - DNA, 41 bp, Artificial Sequence
  Description of Artificial Sequence Synthetic primer
ctgccacacgcccattcttaatataacctttcatcaacgtc

SEQ ID NO: 32 - DNA, 42 bp, Artificial Sequence
  Description of Artificial Sequence Synthetic primer
gttgatgaaaggttatatggcaaatgggcgtgtggcagacac

SEQ ID NO: 33 - DNA, 42 bp, Artificial Sequence
  Description of Artificial Sequence Synthetic primer
gtgtctgccacacgcccatttgccatataacctttcatcaac

SEQ ID NO: 34 - DNA, 42 bp, Artificial Sequence
  Description of Artificial Sequence Synthetic primer
gttgatgaaaggttatatgcacaatgggcgtgtggcagacac

SEQ ID NO: 35 - DNA, 42 bp, Artificial Sequence
  Description of Artificial Sequence Synthetic primer
gtgtctgccacacgcccattgtgcatataacctttcatcaac

SEQ ID NO: 36 - DNA, 42 bp, Artificial Sequence
  Description of Artificial Sequence Synthetic primer
gttgatgaaaggttatatgaataatgggcgtgtggcagacac

SEQ ID NO: 37 - DNA, 42 bp, Artificial Sequence
  Description of Artificial Sequence Synthetic primer
gtgtctgccacacgcccattattcatataacctttcatcaac

SEQ ID NO: 38 - DNA, 42 bp, Artificial Sequence
  Description of Artificial Sequence Synthetic primer
gatgaaaggttatatgaaggcggggcgtgtggcagacacagc

SEQ ID NO: 39 - DNA, 42 bp, Artificial Sequence
  Description of Artificial Sequence Synthetic primer
gctgtgtctgccacacgccccgccttcatataacctttcatc

SEQ ID NO: 40 - DNA, 42 bp, Artificial Sequence
  Description of Artificial Sequence Synthetic primer
gatgaaaggttatatgaagctggggcgtgtggcagacacagc

SEQ ID NO: 41 - DNA, 42 bp, Artificial Sequence
  Description of Artificial Sequence Synthetic primer
gctgtgtctgccacacgccccagcttcatataacctttcatc

SEQ ID NO: 42 - DNA, 42 bp, Artificial Sequence
  Description of Artificial Sequence Synthetic primer
gaaaggttatatgaagaatgcgcgtgtggcagacacagctag

SEQ ID NO: 43 - DNA, 42 bp, Artificial Sequence
  Description of Artificial Sequence Synthetic primer
ctagctgtgtctgccacacgcgcattcttcatataacctttc

SEQ ID NO: 44 - DNA, 42 bp, Artificial Sequence
  Description of Artificial Sequence Synthetic primer
gaaaggttatatgaagaatgatcgtgtggcagacacagctag

SEQ ID NO: 45 - DNA, 42 bp, Artificial Sequence
  Description of Artificial Sequence Synthetic primer
ctagctgtgtctgccacacgatcattcttcatataacctttc

SEQ ID NO: 46 - DNA, 41 bp, Artificial Sequence
  Description of Artificial Sequence Synthetic primer
cccagatgaagttacatacaatacggttgtgtcagcttttg

SEQ ID NO: 47 - DNA, 41 bp, Artificial Sequence
  Description of Artificial Sequence Synthetic primer
caaaagctgacacaaccgtattgtatgtaacttcatctggg

SEQ ID NO: 48 - DNA, 41 bp, Artificial Sequence
  Description of Artificial Sequence Synthetic primer
cccagatgaagttacatacgcaacggttgtgtcagcttttg

SEQ ID NO: 49 - DNA, 41 bp, Artificial Sequence
  Description of Artificial Sequence Synthetic primer
caaaagctgacacaaccgttgcgtatgtaacttcatctggg

SEQ ID NO: 50 - DNA, 43 bp, Artificial Sequence
  Description of Artificial Sequence Synthetic primer
ttacatacactacggttgtggcagcttttgtaaatgcagggtt

SEQ ID NO: 51 - DNA, 43 bp, Artificial Sequence
  Description of Artificial Sequence Synthetic primer
aaccctgcatttacaaaagctgccacaaccgtagtgtatgtaa

SEQ ID NO: 52 - DNA, 43 bp, Artificial Sequence
  Description of Artificial Sequence Synthetic primer
ttacatacactacggttgtgaaagcttttgtaaatgcagggtt

SEQ ID NO: 53 - DNA, 43 bp, Artificial Sequence
  Description of Artificial Sequence Synthetic primer
aaccctgcatttacaaaagctttcacaaccgtagtgtatgtaa

SEQ ID NO: 54 - DNA, 41 bp, Artificial Sequence
  Description of Artificial Sequence Synthetic primer
ggttgtgtcagcttttgtagcagcagggttgatggatagag

SEQ ID NO: 55 - DNA, 41 bp, Artificial Sequence
  Description of Artificial Sequence Synthetic primer
ctctatccatcaaccctgctgctacaaaagctgacacaacc

SEQ ID NO: 56 - DNA, 41 bp, Artificial Sequence
  Description of Artificial Sequence Synthetic primer
ggttgtgtcagcttttgtacgtgcagggttgatggatagag

SEQ ID NO: 57 - DNA, 41 bp, Artificial Sequence
  Description of Artificial Sequence Synthetic primer
ctctatccatcaaccctgcacgtacaaaagctgacacaacc

SEQ ID NO: 58 - DNA, 34 bp, Artificial Sequence
  Description of Artificial Sequence Synthetic primer
cgcactagtcgtacatacactacggttgtgtcag

SEQ ID NO: 59 - DNA, 43 bp, Artificial Sequence
  Description of Artificial Sequence Synthetic primer
ctgcaaataggattacttataccgttctgctcaaaggatattg

SEQ ID NO: 60 - DNA, 43 bp, Artificial Sequence
  Description of Artificial Sequence Synthetic primer
caatatcctttgagcagaacggtataagtaatcctatttgcag

SEQ ID NO: 61 - DNA, 43 bp, Artificial Sequence; identical to SEQ ID NO: 52
  Description of Artificial Sequence Synthetic primer

SEQ ID NO: 62 - DNA, 43 bp, Artificial Sequence; identical to SEQ ID NO: 53
  Description of Artificial Sequence Synthetic primer

SEQ ID NO: 63 - DNA, 43 bp, Artificial Sequence
  Description of Artificial Sequence Synthetic primer
ttacatacactacggttgtggatgcttttgtaaatgcagggtt

SEQ ID NO: 64 - DNA, 43 bp, Artificial Sequence
  Description of Artificial Sequence Synthetic primer
aaccctgcatttacaaaagcatccacaaccgtagtgtatgtaa

SEQ ID NO: 65 - DNA, 44 bp, Artificial Sequence
  Description of Artificial Sequence Synthetic primer
gtttcctataacattataataaaaggatgcattcttatagatga

SEQ ID NO: 66 - DNA, 44 bp, Artificial Sequence
  Description of Artificial Sequence Synthetic primer
tcatctataagaatgcatccttttattataatgttataggaaac

SEQ ID NO: 67 - DNA, 43 bp, Artificial Sequence
  Description of Artificial Sequence Synthetic primer
ttagttacacaactttgatggatgcttttgcaatgtcggggca

SEQ ID NO: 68 - DNA, 43 bp, Artificial Sequence
  Description of Artificial Sequence Synthetic primer
tgccccgacattgcaaaagcatccatcaaagttgtgtaactaa

SEQ ID NO: 69 - DNA, 41 bp, Artificial Sequence
  Description of Artificial Sequence Synthetic primer
ggttgtgtcagcttttgtaaaagcagggttgatggatagag

SEQ ID NO: 70 - DNA, 41 bp, Artificial Sequence
  Description of Artificial Sequence Synthetic primer
ctctatccatcaaccctgcttttacaaaagctgacacaacc

SEQ ID NO: 71 - DNA, 42 bp, Artificial Sequence
  Description of Artificial Sequence Synthetic primer
gttgatgaaaggttatatgatgaatgggcgtgtggcagacac

SEQ ID NO: 72 - DNA, 42 bp, Artificial Sequence
  Description of Artificial Sequence Synthetic primer
gtgtctgccacacgcccattcatcatataacctttcatcaac

SEQ ID NO: 73 - DNA, 35 bp, Artificial Sequence
  Description of Artificial Sequence Synthetic primer
aatacgactcactatagctggatggaatttcagtg

SEQ ID NO: 74 - DNA, 31 bp, Artificial Sequence
  Description of Artificial Sequence Synthetic primer
ccctcgagcgaactaccaaaggagaataggc

SEQ ID NO: 75 - DNA, 2337 bp, Arabidopsis thaliana
atgaacattctccgacctccgacgtcatcatcatcttcgtcgtttcctccatacccaaagcccgtttcattaacccctccggtatctttcactctcatccacaaccccataaacctctgctctataaacccaccattcaccaacgctggtcgaccaattttccaacggtccgcctccggcactgctaatagctccgccgaagacctctcgtctttcttgggctctccctcagaggcgtattcaacacacaacgaccaagagcttttgtttctcctccgcaatagaaaaaccgatgaagcttgggctaagtatgttcaatccactcatctccctggaccaacttgtcttagccgtttagtttctcaattatcttatcaatccaaacccgagagtctcacgcgcgcacaatctatcctcacgcgcctccgcaatgaacgccagctgcatcgccttgacgctaattccctcggtctcctcgccatggctgcagcgaagtctggccaaacactttacgccgtctccgtcatcaagtccatgattcgttctgggtatttacctcatgttaaagcgtggacagctgcagtagctagtctctctgcttccggagatgatggtccggaagaatctatcaaactcttcatcgctattacgcgacgagtcaaacgatttggtgaccagtctttggttggtcaatctaggcctgatacggcggcatttaatgcggtgcttaacgcttgtgctaaccttggtgatactgacaagtattggaagttgttcgaggaaatgtctgagtgggattgtgagcctgatgtcttgacttacaatgttatgattaagctttgtgcgagggttggtcggaaggaattgattgtgtttgtgttggaaaggattattgacaaggggattaaggtttgtatgactacaatgcattctcttgttgcagcttatgttgggtttggagatttgagaactgctgagaggattgttcaagcgatgagggagaaaaggagagatctttgtaaggttctacgagaatgcaacgctgaggatttgaaggagaaagaagaggaagaagcagaagatgatgaagatgcgtttgaggatgatgaagactcgggttattcggctcgggatgaggtaagtgaagagggggttgtagatgtgttcaagaaattgctacctaactcggttgatccgagtggtgagccaccattgttgcctaaagtctttgcaccagactcaaggatctacacgacgttgatgaaaggttatatgaagaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaataggattacttataatgttctgctcaaaggatattgtaagcagttgcagatagatagggcagaggatttactaagagagatgactgaagatgcggggatcgagccagacgtggtttcctataacattataatagatggatgcattcttatagatgatagcgcaggagctctagcgtttttcaatgaaatgagaacgagagggattgcaccaactaagattagttacacaactttgatgaaggcttttgcaatgtcggggcaacccaagttggcgaatagggtgtttgatgagatgatgaatgatccaagggtcaaagttgatttgatcgcgtggaacatgttggttgaagggtactgcaggctaggtttgattgaggatgctcagagagtagtgtcaagaatgaaagaaaacgggttttacccaaatgtggcaacctatgggagtctagccaatggggtttcgcaggcgaggaaacctggtgatgctctcttgctttggaaggagataaaggaaaggtgtgcggtgaaaaagaaagaagcaccttcagattcttcttcagatcctgctcctccgatgctgaaaccagatgaagggttgttagatacactagcggatatatgtgtcagggctgcttttttcaagaaggcattggagataatcgcatgtatggaggagaatgggatacctccgaataagactaagtacaagaagatctatgtggagatgcactcgaggatgttcactagcaaacatgcttcacaagccagaatagataggcgggtagaacgaaagagagcggctgaagctttcaagttttggctcggtttgcctaattcttattatggaagtgaatggaagttaggtccaagagaagactag

SEQ ID NO: 76 - protein, 778 aa, Arabidopsis thaliana
MNILRPPTSSSSSSFPPYPKPVSLTPPVSFTLIHNPINLCSINPPFTNAGRPIFQRSASGTANSSAEDLSSFLGSPSEAYSTHNDQELLFLLRNRKTDEAWAKYVQSTHLPGPTCLSRLVSQLSYQSKPESLTRAQSILTRLRNERQLHRLDANSLGLLAMAAAKSGQTLYAVSVIKSMIRSGYLPHVKAWTAAVASLSASGDDGPEESIKLFIAITRRVKRFGDQSLVGQSRPDTAAFNAVLNACANLGDTDKYWKLFEEMSEWDCEPDVLTYNVMIKLCARVGRKELIVFVLERIIDKGIKVCMTTMHSLVAAYVGFGDLRTAERIVQAMREKRRDLCKVLRECNAEDLKEKEEEEAEDDEDAFEDDEDSGYSARDEVSEEGVVDVFKKLLPNSVDPSGEPPLLPKVFAPDSRIYTTLMKGYMKNGRVADTARMLEAMRRQDDRNSHPDEVTYTTVVSAFVNAGLMDRARQVLAEMARMGVPANRITYNVLLKGYCKQLQIDRAEDLLREMTEDAGIEPDVVSYNIIIDGCILIDDSAGALAFFNEMRTRGIAPTKISYTTLMKAFAMSGQPKLANRVFDEMMNDPRVKVDLIAWNMLVEGYCRLGLIEDAQRVVSRMKENGFYPNVATYGSLANGVSQARKPGDALLLWKEIKERCAVKKKEAPSDSSSDPAPPMLKPDEGLLDTLADICVRAAFFKKALEIIACMEENGIPPNKTKYKKIYVEMHSRMFTSKHASQARIDRRVERKRAAEAFKFWLGLPNSYYGSEWKLGPRED

SEQ ID NO: 77 - RNA, 120 nt, Arabidopsis thaliana
cuggauggaauuucagugaauuagacugagaagaaucuugaaguucuagcuuuuagcucgauacaaaaaaguaaaguaugcaggucuaacaauuuuagccuauucuccuuugguaguucg

SEQ ID NO: 78 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMKNGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 79 - DNA, 237 bp, Artificial Sequence
  Description of Artificial Sequence Synthetic polynucleotide
cgcactagtaggatctacacgacgttgatgaaaggttatatgaagaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 80 - protein, 228 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSVTYTTVVSAFVNAGLMDRARQVLAEMARMGVPANRITYNVLLKGYCKQLQIDRAEDLLREMTEDAGIEPDVVDALALKGELEGKPIPNPLLGLDSTRTGHHHHHH

SEQ ID NO: 81 - DNA, 231 bp, Artificial Sequence
  Description of Artificial Sequence Synthetic polynucleotide
cgcactagtgttacatacactacggttgtgtcagcttttgtaaatgcagggttgatggatagagcaagacaagtgttagccgagatggctcggatgggtgttcctgcaaataggattacttataatgttctgctcaaaggatattgtaagcagttgcagatagatagggcagaggatttactaagagagatgactgaagatgcggggatcgagccagacgtggtcgacgcg

SEQ ID NO: 82 - protein, 228 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSITYNVLLKGYCKQLQIDRAEDLLREMTEDAGIEPDVVSYNIIIDGCILIDDSAGALAFFNEMRTRGIAPTKVDALALKGELEGKPIPNPLLGLDSTRTGHHHHHH

SEQ ID NO: 83 - DNA, 231 bp, Artificial Sequence
  Description of Artificial Sequence Synthetic polynucleotide
cgcactagtattacttataatgttctgctcaaaggatattgtaagcagttgcagatagatagggcagaggatttactaagagagatgactgaagatgcggggatcgagccagacgtggtttcctataacattataatagatggatgcattcttatagatgatagcgcaggagctctagcgtttttcaatgaaatgagaacgagagggattgcaccaactaaggtcgacgcg

SEQ ID NO: 84 - protein, 227 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSVSYNIIIDGCILIDDSAGALAFFNEMRTRGIAPTKISYTTLMKAFAMSGQPKLANRVFDEMMNDPRVKVDVDALALKGELEGKPIPNPLLGLDSTRTGHHHHHH

SEQ ID NO: 85 - DNA, 228 bp, Artificial Sequence
  Description of Artificial Sequence Synthetic polynucleotide
cgcactagtgtttcctataacattataatagatggatgcattcttatagatgatagcgcaggagctctagcgtttttcaatgaaatgagaacgagagggattgcaccaactaagattagttacacaactttgatgaaggcttttgcaatgtcggggcaacccaagttggcgaatagggtgtttgatgagatgatgaatgatccaagggtcaaagttgatgtcgacgcg

SEQ ID NO: 86 - protein, 228 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSISYTTLMKAFAMSGQPKLANRVFDEMMNDPRVKVDLIAWNMLVEGYCRLGLIEDAQRVVSRMKENGFYPNVVDALALKGELEGKPIPNPLLGLDSTRTGHHHHHH

SEQ ID NO: 87 - DNA, 231 bp, Artificial Sequence
  Description of Artificial Sequence Synthetic polynucleotide
cgcactagtattagttacacaactttgatgaaggcttttgcaatgtcggggcaacccaagttggcgaatagggtgtttgatgagatgatgaatgatccaagggtcaaagttgatttgatcgcgtggaacatgttggttgaagggtactgcaggctaggtttgattgaggatgctcagagagtagtgtcaagaatgaaagaaaacgggttttacccaaatgtggtcgacgcg

SEQ ID NO: 88 - protein, 227 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSIAWNMLVEGYCRLGLIEDAQRVVSRMKENGFYPNVATYGSLANGVSQARKPGDALLLWKEIKERCAVKKKVDALALKGELEGKPIPNPLLGLDSTRTGHHHHHH

SEQ ID NO: 89 - DNA, 228 bp, Artificial Sequence
  Description of Artificial Sequence Synthetic polynucleotide
cgcactagtatcgcgtggaacatgttggttgaagggtactgcaggctaggtttgattgaggatgctcagagagtagtgtcaagaatgaaagaaaacgggttttacccaaatgtggcaacctatgggagtctagccaatggggtttcgcaggcgaggaaacctggtgatgctctcttgctttggaaggagataaaggaaaggtgtgcggtgaaaaagaaagtcgacgcg

SEQ ID NO: 90 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSAIYTTLMKGYMKNGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 91 - DNA, 237 bp, Artificial Sequence
  Description of Artificial Sequence Synthetic polynucleotide
cgcactagtgcgatctacacgacgttgatgaaaggttatatgaagaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 92 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSIIYTTLMKGYMKNGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 93 - DNA, 237 bp, Artificial Sequence
  Description of Artificial Sequence Synthetic polynucleotide
cgcactagtattatctacacgacgttgatgaaaggttatatgaagaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 94 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYETLMKGYMKNGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 95 - DNA, 237 bp, Artificial Sequence
  Description of Artificial Sequence Synthetic polynucleotide
cgcactagtaggatctacgaaacgttgatgaaaggttatatgaagaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 96 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYNTLMKGYMKNGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 97 - DNA, 237 bp, Artificial Sequence
  Description of Artificial Sequence Synthetic polynucleotide
cgcactagtaggatctacaacacgttgatgaaaggttatatgaagaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 98 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTILMKGYMKNGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 99 - DNA, 237 bp, Artificial Sequence
  Description of Artificial Sequence Synthetic polynucleotide
cgcactagtaggatctacacgattttgatgaaaggttatatgaagaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 100 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMNGYMKNGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 101 - DNA, 237 bp, Artificial Sequence
  Description of Artificial Sequence Synthetic polynucleotide
cgcactagtaggatctacacgacgttgatgaatggttatatgaagaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 102 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMAGYMKNGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 103 - DNA, 237 bp, Artificial Sequence
  Description of Artificial Sequence Synthetic polynucleotide
cgcactagtaggatctacacgacgttgatggcaggttatatgaagaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 104 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKLYMKNGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 105 - DNA, 237 bp, Artificial Sequence
  Description of Artificial Sequence Synthetic polynucleotide
cgcactagtaggatctacacgacgttgatgaaactgtatatgaagaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 106 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKAYMKNGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 107 - DNA, 237 bp, Artificial Sequence
  Description of Artificial Sequence Synthetic polynucleotide
cgcactagtaggatctacacgacgttgatgaaagcgtatatgaagaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 108 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYAKNGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 109 - DNA, 237 bp, Artificial Sequence
  Description of Artificial Sequence Synthetic polynucleotide
cgcactagtaggatctacacgacgttgatgaaaggttatgcgaagaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 110 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYIKNGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 111 - DNA, 237 bp, Artificial Sequence
  Description of Artificial Sequence Synthetic polynucleotide
cgcactagtaggatctacacgacgttgatgaaaggttatattaagaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 112 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMANGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 113 - DNA, 237 bp, Artificial Sequence
  Description of Artificial Sequence Synthetic polynucleotide
cgcactagtaggatctacacgacgttgatgaaaggttatatggcaaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 114 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMHNGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 115 - DNA, 237 bp, Artificial Sequence
  Description of Artificial Sequence Synthetic polynucleotide
cgcactagtaggatctacacgacgttgatgaaaggttatatgcacaatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 116 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMNNGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 117 - DNA, 237 bp, Artificial Sequence
  Description of Artificial Sequence Synthetic polynucleotide
cgcactagtaggatctacacgacgttgatgaaaggttatatgaataatgggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 118 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMKAGRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 119 - DNA, 237 bp, Artificial Sequence
  Description of Artificial Sequence Synthetic polynucleotide
cgcactagtaggatctacacgacgttgatgaaaggttatatgaaggcggggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 120 - protein, 230 aa, Artificial Sequence; identical to SEQ ID NO: 78
  Description of Artificial Sequence Synthetic polypeptide

SEQ ID NO: 121 - DNA, 237 bp, Artificial Sequence
  Description of Artificial Sequence Synthetic polynucleotide
cgcactagtaggatctacacgacgttgatgaaaggttatatgaagctggggcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 122 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMKNARVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 123 - DNA, 237 bp, Artificial Sequence
  Description of Artificial Sequence Synthetic polynucleotide
cgcactagtaggatctacacgacgttgatgaaaggttatatgaagaatgcgcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 124 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMKNDRVADTARMLEAMRRQDDRNSHPDEVTYTTVVSAFVNAGLMDRARQVLAEMARMGVPANRVDALALKGELEGKPIPNPLLGLDSTRTGHHHHHH

SEQ ID NO: 125 - DNA, 237 bp, Artificial Sequence
  Description of Artificial Sequence Synthetic polynucleotide
cgcactagtaggatctacacgacgttgatgaaaggttatatgaagaatgatcgtgtggcagacacagctagaatgcttgaggcaatgaggcgtcaagatgatagaaacagtcacccagatgaagttacatacactacggttgtgtcagcttttgtaaatgcagggttgatggatagagcaagacaagtgttagccgagatggctcggatgggtgttcctgcaaatagggtcgacgcg

SEQ ID NO: 126 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMKNGRVADTARMLEAMRRQDDRNSHPDEVTYNTVVSAFVNAGLMDRARQVLAEMARMGVPANRVDALALKGELEGKPIPNPLLGLDSTRTGHHHHHH

SEQ ID NO: 127 - DNA, 237 bp, Artificial Sequence
  Description of Artificial Sequence Synthetic polynucleotide
cgcactagtaggatctacacgacgttgatgaaaggttatatgaagaatgggcgtgtggcagacacagctagaatgcttgaggcaatgaggcgtcaagatgatagaaacagtcacccagatgaagttacatacaatacggttgtgtcagcttttgtaaatgcagggttgatggatagagcaagacaagtgttagccgagatggctcggatgggtgttcctgcaaatagggtcgacgcg

SEQ ID NO: 128 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMKNGRVADTARMLEAMRRQDDRNSHPDEVTYATVVSAFVNAGLMDRARQVLAEMARMGVPANRVDALALKGELEGKPIPNPLLGLDSTRTGHHHHHH

SEQ ID NO: 129 - DNA, 237 bp, Artificial Sequence
  Description of Artificial Sequence Synthetic polynucleotide
cgcactagtaggatctacacgacgttgatgaaaggttatatgaagaatgggcgtgtggcagacacagctagaatgcttgaggcaatgaggcgtcaagatgatagaaacagtcacccagatgaagttacatacgcaacggttgtgtcagcttttgtaaatgcagggttgatggatagagcaagacaagtgttagccgagatggctcggatgggtgttcctgcaaatagggtcgacgcg

SEQ ID NO: 130 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMKNGRVADTARMLEAMRRQDDRNSHPDEVTYTTVVAAFVNAGLMDRARQVLAEMARMGVPANRVDALALKGELEGKPIPNPLLGLDSTRTGHHHHHH

SEQ ID NO: 131 - DNA, 237 bp, Artificial Sequence
  Description of Artificial Sequence Synthetic polynucleotide
cgcactagtaggatctacacgacgttgatgaaaggttatatgaagaatgggcgtgtggcagacacagctagaatgcttgaggcaatgaggcgtcaagatgatagaaacagtcacccagatgaagttacatacactacggttgtggcagcttttgtaaatgcagggttgatggatagagcaagacaagtgttagccgagatggctcggatgggtgttcctgcaaatagggtcgacgcg

SEQ ID NO: 132 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMKNGRVADTARMLEAMRRQDDRNSHPDEVTYTTVVKAFVNAGLMDRARQVLAEMARMGVPANRVDALALKGELEGKPIPNPLLGLDSTRTGHHHHHH

SEQ ID NO: 133 - DNA, 237 bp, Artificial Sequence
  Description of Artificial Sequence Synthetic polynucleotide
cgcactagtaggatctacacgacgttgatgaaaggttatatgaagaatgggcgtgtggcagacacagctagaatgcttgaggcaatgaggcgtcaagatgatagaaacagtcacccagatgaagttacatacactacggttgtgaaagcttttgtaaatgcagggttgatggatagagcaagacaagtgttagccgagatggctcggatgggtgttcctgcaaatagggtcgacgcg

SEQ ID NO: 134 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMKNGRVADTARMLEAMRRQDDRNSHPDEVTYTTVVSAFVAAGLMDRARQVLAEMARMGVPANRVDALALKGELEGKPIPNPLLGLDSTRTGHHHHHH

SEQ ID NO: 135 - DNA, 237 bp, Artificial Sequence
  Description of Artificial Sequence Synthetic polynucleotide
cgcactagtaggatctacacgacgttgatgaaaggttatatgaagaatgggcgtgtggcagacacagctagaatgcttgaggcaatgaggcgtcaagatgatagaaacagtcacccagatgaagttacatacactacggttgtgtcagcttttgtagcagcagggttgatggatagagcaagacaagtgttagccgagatggctcggatgggtgttcctgcaaatagggtcgacgcg

SEQ ID NO: 136 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMKNGRVADTARMLEAMRRQDDRNSHPDEVTYTTVVSAFVRAGLMDRARQVLAEMARMGVPANRVDALALKGELEGKPIPNPLLGLDSTRTGHHHHHH

SEQ ID NO: 137 - DNA, 237 bp, Artificial Sequence
  Description of Artificial Sequence Synthetic polynucleotide
cgcactagtaggatctacacgacgttgatgaaaggttatatgaagaatgggcgtgtggcagacacagctagaatgcttgaggcaatgaggcgtcaagatgatagaaacagtcacccagatgaagttacatacactacggttgtgtcagcttttgtacgtgcagggttgatggatagagcaagacaagtgttagccgagatggctcggatgggtgttcctgcaaatagggtcgacgcg

SEQ ID NO: 138 - protein, 228 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRTYTTVVSAFVNAGLMDRARQVLAEMARMGVPANRITYNVLLKGYCKQLQIDRAEDLLREMTEDAGIEPDVVDALALKGELEGKPIPNPLLGLDSTRTGHHHHHH

SEQ ID NO: 139 - DNA, 231 bp, Artificial Sequence
  Description of Artificial Sequence Synthetic polynucleotide
cgcactagtcgtacatacactacggttgtgtcagcttttgtaaatgcagggttgatggatagagcaagacaagtgttagccgagatggctcggatgggtgttcctgcaaataggattacttataatgttctgctcaaaggatattgtaagcagttgcagatagatagggcagaggatttactaagagagatgactgaagatgcggggatcgagccagacgtggtcgacgcg

SEQ ID NO: 140 - protein, 228 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSVTYTTVVSAFVNAGLMDRARQVLAEMARMGVPANRITYTVLLKGYCKQLQIDRAEDLLREMTEDAGIEPDVVDALALKGELEGKPIPNPLLGLDSTRTGHHHHHH

SEQ ID NO: 141 - DNA, 231 bp, Artificial Sequence
  Description of Artificial Sequence Synthetic polynucleotide
cgcactagtgttacatacactacggttgtgtcagcttttgtaaatgcagggttgatggatagagcaagacaagtgttagccgagatggctcggatgggtgttcctgcaaataggattacttataccgttctgctcaaaggatattgtaagcagttgcagatagatagggcagaggatttactaagagagatgactgaagatgcggggatcgagccagacgtggtcgacgcg

SEQ ID NO: 142 - protein, 228 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSVTYTTVVKAFVNAGLMDRARQVLAEMARMGVPANRITYNVLLKGYCKQLQIDRAEDLLREMTEDAGIEPDVVDALALKGELEGKPIPNPLLGLDSTRTGHHHHHH

SEQ ID NO: 143 - DNA, 231 bp, Artificial Sequence
  Description of Artificial Sequence Synthetic polynucleotide
cgcactagtgttacatacactacggttgtgaaagcttttgtaaatgcagggttgatggatagagcaagacaagtgttagccgagatggctcggatgggtgttcctgcaaataggattacttataatgttctgctcaaaggatattgtaagcagttgcagatagatagggcagaggatttactaagagagatgactgaagatgcggggatcgagccagacgtggtcgacgcg

SEQ ID NO: 144 - protein, 228 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSVTYTTVVDAFVNAGLMDRARQVLAEMARMGVPANRITYNVLLKGYCKQLQIDRAEDLLREMTEDAGIEPDVVDALALKGELEGKPIPNPLLGLDSTRTGHHHHHH

SEQ ID NO: 145 - DNA, 231 bp, Artificial Sequence
  Description of Artificial Sequence Synthetic polynucleotide
cgcactagtgttacatacactacggttgtggatgcttttgtaaatgcagggttgatggatagagcaagacaagtgttagccgagatggctcggatgggtgttcctgcaaataggattacttataatgttctgctcaaaggatattgtaagcagttgcagatagatagggcagaggatttactaagagagatgactgaagatgcggggatcgagccagacgtggtcgacgcg

SEQ ID NO: 146 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMNNGRVADTARMLEAMRRQDDRNSHPDEVTYTTVVSAFVKAGLMDRARQVLAEMARMGVPANRVDALALKGELEGKPIPNPLLGLDSTRTGHHHHHH

SEQ ID NO: 147 - DNA, 237 bp, Artificial Sequence
  Description of Artificial Sequence Synthetic polynucleotide
cgcactagtaggatctacacgacgttgatgaaaggttatatgaataatgggcgtgtggcagacacagctagaatgcttgaggcaatgaggcgtcaagatgatagaaacagtcacccagatgaagttacatacactacggttgtgtcagcttttgtacgtgcagggttgatggatagagcaagacaagtgttagccgagatggctcggatgggtgttcctgcaaatagggtcgacgcg

SEQ ID NO: 148 - protein, 230 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSRIYTTLMKGYMMNGRVADTARMLEAMRRQDDRNSHPDEVTYTTVVSAFVRAGLMDRARQVLAEMARMGVPANRVDALALKGELEGKPIPNPLLGLDSTRTGHHHHHH

SEQ ID NO: 149 - DNA, 237 bp, Artificial Sequence
  Description of Artificial Sequence Synthetic polynucleotide
cgcactagtaggatctacacgacgttgatgaaaggttatatgatgaatgggcgtgtggcagacacagctagaatgcttgaggcaatgaggcgtcaagatgatagaaacagtcacccagatgaagttacatacactacggttgtgtcagcttttgtacgtgcagggttgatggatagagcaagacaagtgttagccgagatggctcggatgggtgttcctgcaaatagggtcgacgcg

SEQ ID NO: 150 - protein, 227 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSVSYNIIIKGCILIDDSAGALAFFNEMRTRGIAPTKISYTTLMKAFAMSGQPKLANRVFDEMMNDPRVKVDVDALALKGELEGKPIPNPLLGLDSTRTGHHHHHH

SEQ ID NO: 151 - DNA, 228 bp, Artificial Sequence
  Description of Artificial Sequence Synthetic polynucleotide
cgcactagtgtttcctataacattataataaaaggatgcattcttatagatgatagcgcaggagctctagcgtttttcaatgaaatgagaacgagagggattgcaccaactaagattagttacacaactttgatgaaggcttttgcaatgtcggggcaacccaagttggcgaatagggtgtttgatgagatgatgaatgatccaagggtcaaagttgatgtcgacgcg

SEQ ID NO: 152 - protein, 227 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSVSYNIIIDGCILIDDSAGALAFFNEMRTRGIAPTKISYTTLMDAFAMSGQPKLANRVFDEMMNDPRVKVDVDALALKGELEGKPIPNPLLGLDSTRTGHHHHHH

SEQ ID NO: 153 - DNA, 228 bp, Artificial Sequence
  Description of Artificial Sequence Synthetic polynucleotide
cgcactagtgtttcctataacattataatagatggatgcattcttatagatgatagcgcaggagctctagcgtttttcaatgaaatgagaacgagagggattgcaccaactaagattagttacacaactttgatggatgcttttgcaatgtcggggcaacccaagttggcgaatagggtgtttgatgagatgatgaatgatccaagggtcaaagttgatgtcgacgcg

SEQ ID NO: 154 - protein, 227 aa, Artificial Sequence
  Description of Artificial Sequence Synthetic polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSVSYNIIIKGCILIDDSAGALAFFNEMRTRGIAPTKISYTTLMDAFAMSGQPKLANRVFDEMMNDPRVKVDVDALALKGELEGKPIPNPLLGLDSTRTGHHHHHH

SEQ ID NO: 155 - DNA, 228 bp, Artificial Sequence
  Description of Artificial Sequence Synthetic polynucleotide
cgcactagtgtttcctataacattataataaaaggatgcattcttatagatgatagcgcaggagctctagcgtttttcaatgaaatgagaacgagagggattgcaccaactaagattagttacacaactttgatggatgcttttgcaatgtcggggcaacccaagttggcgaatagggtgtttgatgagatgatgaatgatccaagggtcaaagttgatgtcgacgcg

SEQ ID NO: 156 - protein, 35 aa, Arabidopsis thaliana
ETYNRMIKVFCESGSASSSYSIVAEMERKGIKPNS

SEQ ID NO: 157 - RNA, 29 nt, Artificial Sequence
  Description of Artificial Sequence Synthetic oligonucleotide
aucgaaaaaaaaaaaaaaaaaaaaaaaaa

SEQ ID NO: 158 - RNA, 29 nt, Artificial Sequence
  Description of Artificial Sequence Synthetic oligonucleotide
aucguuuuuuuuuuuuuuuuuuuuuuuuu

SEQ ID NO: 159 - RNA, 29 nt, Artificial Sequence
  Description of Artificial Sequence Synthetic oligonucleotide
aucgggggggggggggggggggggggggg

SEQ ID NO: 160 - RNA, 29 nt, Artificial Sequence
  Description of Artificial Sequence Synthetic oligonucleotide
aucgccccccccccccccccccccccccc

SEQ ID NO: 161 - DNA, 34 bp, Artificial Sequence
  Description of Artificial Sequence Synthetic primer
cgcactagtgcggcatttaatgcggtgcttaacg

SEQ ID NO: 162 - DNA, 33 bp, Artificial Sequence
  Description of Artificial Sequence Synthetic primer
cgcgtcgaccatacaaaccttaatccccttgtc

SEQ ID NO: 163 - DNA, 34 bp, Artificial Sequence
  Description of Artificial Sequence Synthetic primer
cgcactagtttgacttacaatgttatgattaagc

SEQ ID NO: 164 - DNA, 34 bp, Artificial Sequence
  Description of Artificial Sequence Synthetic primer
cgcgtcgaccttacaaagatctctccttttctcc

SEQ ID NO: 165 - DNA, 33 bp, Artificial Sequence
  Description of Artificial Sequence Synthetic primer
cgcactagtgacttgatttcgtggaactcaatg

SEQ ID NO: 166 - DNA, 35 bp, Artificial Sequence
  Description of Artificial Sequence Synthetic primer
cgcgtcgactcttctcggcatcacatcgaataaac

SEQ ID NO: 167 - DNA, 33 bp, Artificial Sequence
  Description of Artificial Sequence Synthetic primer
cgcactagtgacttgttatcgtggaactcaatg

SEQ ID NO: 168 - protein, 227 aa, Artificial Sequence
  Description of Artificial Sequence Synthetic HCF152/2&3 polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSAAFNAVLNACANLGDTDKYWKLFEEMSEWDCEPDVLTYNVMIKLCARVGRKELIVFVLERIIDKGIKVCMVDALALKGELEGKPIPNPLLGLDSTRTGHHHHHH

SEQ ID NO: 169 - DNA, 228 bp, Artificial Sequence
  Description of Artificial Sequence Synthetic HCF152/2&3 polynucleotide
cgcactagtgcggcatttaatgcggtgcttaacgcttgtgctaaccttggtgatactgacaagtattggaagttgttcgaggaaatgtctgagtgggattgtgagcctgatgtcttgacttacaatgttatgattaagctttgtgcgagggttggtcggaaggaattgattgtgtttgtgttggaaaggattattgacaaggggattaaggtttgtatggtcgacgcg

SEQ ID NO: 170 - protein, 227 aa, Artificial Sequence
  Description of Artificial Sequence Synthetic HCF152/3&4 polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSLTYNVMIKLCARVGRKELIVFVLERIIDKGIKVCMTTMHSLVAAYVGFGDLRTAERIVQAMREKRRDLCKVDALALKGELEGKPIPNPLLGLDSTRTGHHHHHH

SEQ ID NO: 171 - DNA, 228 bp, Artificial Sequence
  Description of Artificial Sequence Synthetic HCF152/3&4 polynucleotide
cgcactagtttgacttacaatgttatgattaagctttgtgcgagggttggtcggaaggaattgattgtgtttgtgttggaaaggattattgacaaggggattaaggtttgtatgactacaatgcattctcttgttgcagcttatgttgggtttggagatttgagaactgctgagaggattgttcaagcgatgagggagaaaaggagagatctttgtaaggtcgacgcg

SEQ ID NO: 172 - protein, 188 aa, Artificial Sequence
  Description of Artificial Sequence Synthetic CRR4/6 polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSDLISWNSMIDGYVKHGRIEDAKGLFDVMPRRVDALALKGELEGKPIPNPLLGLDSTRTGHHHHHH

SEQ ID NO: 173 - DNA, 111 bp, Artificial Sequence
  Description of Artificial Sequence Synthetic CRR4/6 polynucleotide
cgcactagtgacttgatttcgtggaactcaatgatagatggatatgtaaaacacggaagaatcgaagatgctaagggtttattcgatgtgatgccgagaagagtcgacgcg

SEQ ID NO: 174 - protein, 188 aa, Artificial Sequence
  Description of Artificial Sequence Synthetic CRR4/6(I1A) polypeptide
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKRTSDLLSWNSMIDGYVKHGRIEDAKGLFDVMPRRVDALALKGELEGKPIPNPLLGLDSTRTGHHHHHH

SEQ ID NO: 175 - DNA, 111 bp, Artificial Sequence; identical to SEQ ID NO: 173
  Description of Artificial Sequence Synthetic CRR4/6(I1A) polynucleotide

SEQ ID NO: 176 - DNA, 1842 bp, Unknown
  Description of Unknown CRR4(at2g45350) sequence
atgcttgtcttcaagtcaaccatggagtgttcgatttcatccaccattcatgtccttggaagctgcaaaacttcagatgacgtgaaccaaatccacgggcgattgattaagacgggaatcatcaaaaactcaaatctcactacgaggattgttctggcttttgcctcttctcgacgtccgtatctcgccgatttcgcgcgttgtgtcttccacgagtatcacgtatgttcgttttcatttggagaggtggaggatccatttttatggaacgccgtgatcaagtctcactctcatggaaaggatccgagacaagctctgctcttgctctgtttgatgctcgagaatggggtttccgtggacaaattctcactgtcacttgttcttaaagcgtgttcgaggttaggttttgtaaaaggaggaatgcagattcatgggtttttgaaaaaaactggactttggtcggatttgtttctacagaattgtttgattggcttgtatctgaaatgtggttgtttaggtttatcacgccagatgtttgatagaatgccgaagagagactctgtttcttataattccatgattgatgggtatgtcaaatgtggattgattgtatccgcgcgtgaattgttcgatttgatgcctatggagatgaagaatttgatatcttggaactctatgataagtggttatgctcagacatcagatggagttgacatagcgtctaaactgtttgctgatatgcctgagaaggacttgatttcgtggaactcaatgatagatggatatgtaaaacacggaagaatcgaagatgctaagggtttattcgatgtgatgccgagaagagatgtagttacttgggctaccatgattgatgggtatgcaaagttaggttttgttcatcacgctaagactctgtttgaccaaatgcctcatagagatgttgtggcatataattctatgatggctggttatgttcaaaacaagtatcacatggaagctcttgaaatatttagtgacatggaaaaggagagtcacttgttacccgatgatacgactttggtaatagttctgcctgcaattgctcagcttggccgattatccaaagccatagatatgcatttgtacatcgtggagaaacaattctatctaggtggaaaactcggtgttgctctcattgatatgtattcgaaatgcggaagcatacaacacgccatgttggttttcgagggaatcgaaaacaaaagcattgatcactggaatgctatgattggtgggctcgctattcatggtctaggggaatctgcattcgatatgctcttgcagattgagagactctctttaaaaccagacgatatcacctttgttggcgttttaaatgcttgcagccactctgggttagtaaaggaaggccttctctgctttgagctcatgaggagaaaacacaagatagaaccaagattgcaacactatggttgtatggtagacatactatcgagatccggaagtatagagctagccaaaaacttaatagaggaaatgcctgttgagccaaatgatgtcatatggagaacgtttctcaccgcttgtagtcaccacaaggagtttgaaacgggagagcttgtcgcaaaacaccttattttgcaggctggatataacccgagctcatatgtgctactctctaacatgtatgctagttttggaatgtggaaggatgttcgtagagttagaacgatgatgaaggaaagaaagatagagaaaattcctggttgtagttggattgagctcgatggaagagtccatgagttctttgtagatagcattgaagtttccagtacattgtag

SEQ ID NO: 177 - protein, 613 aa, Unknown
  Description of Unknown CRR4(at2g45350) sequence
MLVFKSTMECSISSTIHVLGSCKTSDDVNQIHGRLIKTGIIKNSNLTTRIVLAFASSRRPYLADFARCVFHEYHVCSFSFGEVEDPFLWNAVIKSHSHGKDPRQALLLLCLMLENGVSVDKFSLSLVLKACSRLGFVKGGMQIHGFLKKTGLWSDLFLQNCLIGLYLKCGCLGLSRQMFDRMPKRDSVSYNSMIDGYVKCGLIVSARELFDLMPMEMKNLISWNSMISGYAQTSDGVDIASKLFADMPEKDLISWNSMIDGYVKHGRIEDAKGLFDVMPRRDVVTWATMIDGYAKLGFVHHAKTLFDQMPHRDVVAYNSMMAGYVQNKYHMEALEIFSDMEKESHLLPDDTTLVIVLPAIAQLGRLSKAIDMHLYIVEKQFYLGGKLGVALIDMYSKCGSIQHAMLVFEGIENKSIDHWNAMIGGLAIHGLGESAFDMLLQIERLSLKPDDITFVGVLNACSHSGLVKEGLLCFELMRRKHKIEPRLQHYGCMVDILSRSGSIELAKNLIEEMPVEPNDVIWRTFLTACSHHKEFETGELVAKHLILQAGYNPSSYVLLSNMYASFGMWKDVRRVRTMMKERKIEKIPGCSWIELDGRVHEFFVDSIEVSSTL